(19) United States
(12) Patent Application Publication
(10) Pub. No.: US 2019/0233850 A1
(43) Pub. Date: Aug. 1, 2019

(54) METHODS AND MATERIALS FOR THE BIOSYNTHESIS OF BETA HYDROXY ACIDS AND DERIVATIVES AND COMPOUNDS RELATED THERETO

(71) Applicant: INVISTA NORTH AMERICA S.A.R.L., Wilmington, DE (US)

(72) Inventors: Ana Teresa dos Santos Brito Mendes ROBERTS, Redcar (GB); Alexander Brett FOSTER, Redcar (GB)

(21) Appl. No.: 16/264,759

(22) Filed: Feb. 1, 2019

Related U.S. Application Data
(60) Provisional application No. 62/659,288, filed on Apr. 18, 2018.
(60) Provisional application No. 62/625,047, filed on Feb. 1, 2018.
(60) Provisional application No. 62/624,885, filed on Feb. 1, 2018.

(51) Int. Cl. C12P 7/42; C12N 15/74; C12N 9/02

(52) U.S. Cl. CPC C12P 7/42; C12N 9/0008; C12Y 102/01075; C12N 15/74

(57) ABSTRACT

Methods and materials for the production of beta hydroxy acids, such as 3-hydroxypropanoic acid (3-HP) and derivatives and compounds related thereto are provided. Also provided are products produced in accordance with these methods and materials.

This patent application claims the benefit of priority of U.S. Provisional Application Ser. No. 62/659,288 filed Apr. 18, 2018, U.S. Provisional Application Ser. No. 62/625,047 filed Feb. 1, 2018 and U.S. Provisional Application Ser. No. 62/624,885 filed Feb. 1, 2018, the contents of each of which are herein incorporated by reference in their entireties.

### FIELD

The present invention relates to biosynthetic methods and materials for the production of beta hydroxy acids, such as 3-hydroxypropanoic acid (3-HP), and/or derivatives thereof and/or other compounds related thereto. The present invention also relates to products biosynthesized or otherwise encompassed by these methods and materials.

Replacement of traditional chemical production processes relying on, for example fossil fuels and/or potentially toxic chemicals, with environmentally friendly (e.g., green chemicals) and/or “cleantech” solutions is being considered, including work to identify suitable building blocks for such use in the manufacturing of such chemicals. See, “Conservative evolution and industrial metabolism in Green Chemistry”, Green Chem., 2018, 20, 2171-2191.

Beta hydroxy acids, such as 3-HP, have been identified as a value-added platform compound among renewable biomass production products proposed by the United States Department of Energy (Werpy, T. & Petersen, G. US DOE, Washington, D.C., 2004). 3-HP has versatile applications in, for example, but not limited to, conversion to bulk chemicals such as acrylic acid (see WO 2013/192451), 1,3-propanediol, 3-hydroxypropionaldehyde and malonic acid as well as plastics (Valdehuesa et al. Appl. Microbiol. Biotechnol. 2013 97:3309-3321) and in the polymerization and formation biodegradable materials.

Several microbes that are able to naturally produce 3-HP have been identified (Kumar et al. Biotechnol Adv. 2013 31:945-961). However, low yield of 3-HP has reportedly restricted commercialization (Li et al. Scientific Reports 2016 6:26932).

Acetyl-Coenzyme A (CoA) from central metabolism is converted into malonyl-CoA by acetyl-CoA carboxylases (ACC) which can be directed into the fatty acid biosynthesis. In the presence of a malonyl-CoA reductase (MCR), malonyl-CoA can be reduced to 3-HP in a two-step reaction (Hügler et al. Journal of Bacteriology 2002 184(9): 2404-10) (See FIG. 1). In the first step malonyl-CoA is reduced by the MCR/C-terminal into malonate semialdehyde. The second step is performed by the MCR/N-terminal and converts the semialdehyde into 3-HP.

This pathway has been engineered in a few organisms such as Escherichia coli (Rathnasingh et al. Journal of Biotechnology 2012 157(4): 633-40; Cheng et al. Bioresource Technology 2016 200:897-904), Saccharomyces cerevisiae (Chen et al. Metabolic Engineering 2014 22: 104-9; Kildegaard et al. Microbial Cell Factories. 2016 15: 53) and Synechocystis sp. (Wang et al. Metabolic Engineering. 2016 34: 60-70).

Further, Liu and co-workers reviewed several strategies previously adopted to engineering the malonyl-CoA pathway as a route to 3-HP biosynthesis including redirection of carbon from pyruvate to malonyl-CoA by manipulating the tricarboxylic acid (TCA) cycle and acetyl-CoA synthetases, redirection of carbon from malonyl-CoA to 3-HP by blocking competitive pathways such as fatty acid synthesis, improving catalysis of key enzymes such as ACC and MCR, and enhancing cofactors and energy supply such as biotin, ATP and NAD(P)H (Critical Reviews in Biotechnology 2017 37(7): 933-941).

Engineering of a pathway in Cupriavidus necator for generation of 3-HP-CoA has also been reported (Fukui et al. Biomacromolecules 2009 10(4):700-6).

Biosynthetic materials and methods, including organisms having increased production of 3-HP, derivatives thereof and compounds related thereto are needed.

### SUMMARY OF THE INVENTION

An aspect of the present invention relates to a process for biosynthesis of 3-HP and/or derivatives thereof and/or compounds related thereto. The process comprises obtaining an organism capable of producing and/or accumulating 3-HP and derivatives and compounds related thereto, altering the organism, and producing and/or accumulating more 3-HP and derivatives and compounds related thereto in the altered organism as compared to the unaltered organism. In one nonlimiting embodiment, the organism is C. necator or an organism with one or more properties similar thereto. In one nonlimiting embodiment, the organism is altered to express malonyl-CoA reductase (MCR). In one nonlimiting embodiment, the MCR comprises Chloroflexus aurantiacus MCR (SEQ ID NO:1) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 1 or a functional fragment thereof. In one nonlimiting embodiment, the MCR is encoded by a nucleic acid sequence comprising Chloroflexus aurantiacus MCR (SEQ ID NO:2) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 2 or a functional fragment thereof. In one nonlimiting embodiment, the MCR is EC 1.2.1.75.

In one nonlimiting embodiment, the nucleic acid sequence is codon optimized for C. necator.

In one nonlimiting embodiment, the organism is further altered to redirect the carbon flux to 3-HP via interference with any one or more of a malonate semialdehyde dehydrogenase such as MMSA1, MMSA2 and/or MMSA3, enzymes that potentially degrade malonate semialdehyde into acetyl-CoA; and/or a malonyl-CoA decarboxylase (MCD) that converts malonyl-CoA back into acetyl-CoA; and/or a 3-hydroxypropionate dehydrogenase (HPDH) that converts 3-HP into malonate semialdehyde; and/or another a 3-hydroxyisobutyrate dehydrogenase (MMSB) that could putatively convert malonate semialdehyde into (S)3-hydroxybutyrate; and/or a 2-hydroxy-3-oxopropionate reductase; and/or a NAD-dependent beta-hydroxyacid dehydrogenase (mmsB), a choline dehydrogenase, a glucose-methanol-choline oxidoreductase and/or a oxidoreductase (hpdH) which convert 3-hydroxypropionate to malonate semialdehyde; and/or a CoA transferase or a CoA ligase which converts 3-hydroxypropionate to 3-hydroxypropionate-CoA; and/or one or more enzymes converting 3-hydroxypropionate to succinyl-CoA as depicted, for example, in FIG. 1.

In one nonlimiting embodiment, the organism is further modified to eliminate phaCAB, involved in PHBs production and/or H16-A0006-9 encoding endonucleases thereby improving transformation efficiency.

Another aspect of the present invention relates to an organism altered to produce and/or accumulates more 3-HP and/or derivatives and compounds related thereto as compared to the unaltered organism. In one nonlimiting embodiment, the organism is C. necator or an organism with properties similar thereto. In one nonlimiting embodiment, the organism is altered to express MCR. In one nonlimiting embodiment, the MCR comprises Chloroflexus aurantiacus MCR (SEQ ID NO:1) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 1 or a functional fragment thereof. In one nonlimiting embodiment, the MCR is encoded by a nucleic acid sequence comprising Chloroflexus aurantiacus MCR (SEQ ID NO:2) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 2 or a functional fragment thereof. In one nonlimiting embodiment, the MCR is EC 1.2.1.75.

In one nonlimiting embodiment, the nucleic acid sequence is codon optimized for C. necator.

In one nonlimiting embodiment, the organism is further altered to redirect the carbon flux to 3-HP via interference with any one or more of a malonate semialdehyde dehydrogenase such as MMSA1, MMSA2 and/or MMSA3, enzymes that potentially degrade malonate semialdehyde into acetyl-CoA; and/or a malonyl-CoA decarboxylase (MCD) that converts malonyl-CoA back into acetyl-CoA; and/or a 3-hydroxypropionate dehydrogenase (HPDH) that converts 3-HP into malonate semialdehyde; and/or another a 3-hydroxyisobutyrate dehydrogenase (MMSB) that could putatively convert malonate semialdehyde into (S)3-hydroxybutyrate; and/or a 2-hydroxy-3-oxopropionate reductase; and/or a NAD-dependent beta-hydroxyacid dehydrogenase (mmsB), a choline dehydrogenase, a glucose-methanol-choline oxidoreductase and/or a oxidoreductase (hpdH) which converts 3-hydroxypropionate to malonate semialdehyde; and/or a CoA transferase or a CoA ligase which converts 3-hydroxypropionate to 3-hydroxypropionate-CoA; and/or one or more enzymes converting 3-hydroxypropionate to succinyl-CoA as depicted, for example, in FIG. 1.

In one nonlimiting embodiment, the organism is further modified to eliminate phaCAB, involved in PHBs production and/or H16-A0006-9 encoding endonucleases thereby improving transformation efficiency.

In one nonlimiting embodiment, the organism is altered to express, overexpress, not express or express less of one or more molecules depicted in FIG. 1. In one nonlimiting embodiment, the molecule(s) comprise a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence corresponding to a molecule(s) depicted in FIG. 1, or a functional fragment thereof.

Another aspect of the present invention relates to bio-derived, bio-based, or fermentation-derived products produced from any of the methods and/or altered organisms disclosed herein. Such products include compositions comprising at least one bio-derived, bio-based, or fermentation-derived compound or any combination thereof, as well as bio-derived, bio-based, or fermentation-derived polymers comprising these bio-derived, bio-based, or fermentation-derived compositions or compounds; bio-derived, bio-based, or fermentation-derived plastics comprising the bio-derived, bio-based, or fermentation-derived compositions or compounds or any combination thereof or the bio-derived, bio-based, or fermentation-derived polymers or any combination thereof; molded substances obtained by molding the bio-derived, bio-based, or fermentation-derived polymers or the bio-derived, bio-based, or fermentation-derived plastics or any combination thereof; bio-derived, bio-based, or fermentation-derived formulations comprising the bio-derived, bio-based, or fermentation-derived compositions or compounds, polymers or plastics, or the bio-derived, bio-based, or fermentation-derived molded substances, or any combination thereof; and bio-derived, bio-based, or fermentation-derived semi-solids or non-semi-solid streams comprising the bio-derived, bio-based, or fermentation-derived compositions or compounds, polymers, plastics, molded substances or formulations, or any combination thereof.

Another aspect of the present invention relates to a bio-derived, bio-based or fermentation derived product biosynthesized in accordance with the exemplary central metabolism depicted in FIG. 1.

Another aspect of the present invention relates to exogenous genetic molecules of the altered organisms disclosed herein. In one nonlimiting embodiment, the exogenous genetic molecule comprises a codon optimized nucleic acid sequence. In one nonlimiting embodiment, the nucleic acid sequence is codon optimized for C. necator. In one nonlimiting embodiment, the exogenous genetic molecule comprises a nucleic acid sequence comprising Chloroflexus aurantiacus MCR (SEQ ID NO:2) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 2 or a functional fragment thereof. Additional nonlimiting examples of exogenous genetic molecules include expression constructs of, for example, MCR and synthetic operons of, for example MCR.

Yet another aspect of the present invention relates to means and processes for use of these means for biosynthesis of 3-HP including derivatives thereof and/or compounds related thereto.

### BRIEF DESCRIPTION OF THE FIGURES

FIG. 1 is a schematic of the biochemical pathway from acetyl-CoA to 3-hydroxypropionate (3-HP).

FIGS. 2A and 2B are illustrative images of vectors pBBR1(1B)::pBAD::Ca_MCR*::rrnbT1 and pBBR1(1B):pBAD. The nucleic acid sequence of the vector depicted in FIG. 2B is set forth herein in SEQ ID NO: 3.

FIG. 3 is a schematic representation of the oxidative and reductive routes for the degradation of 3-hydroxypropionate.

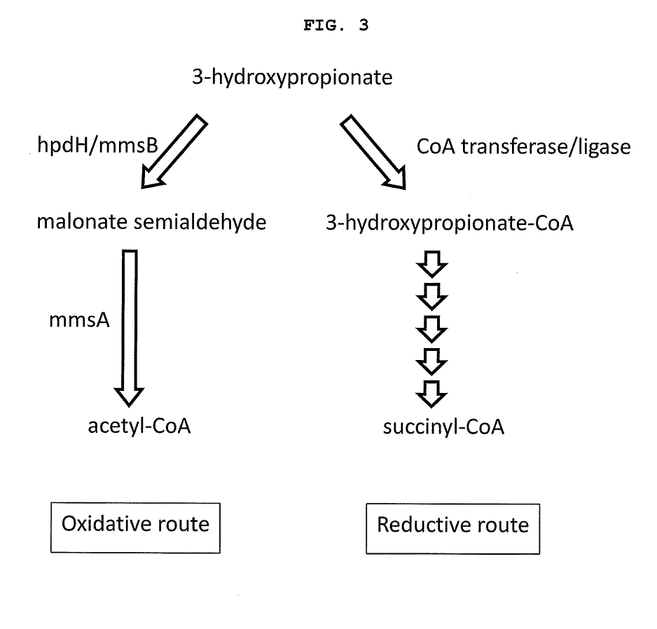

### DETAILED DESCRIPTION

The present invention provides processes for biosynthesis of beta hydroxy acids, such as 3-hydroxypropanoic acid (3-HP), and/or derivatives thereof, and/or compounds related thereto, and organisms altered to increase biosynthesis of 3-HP, derivatives thereof and compounds related thereto, and organisms related thereto, exogenous genetic molecules of these altered organisms, and bio-derived, bio-based, or fermentation-derived products biosynthesized or otherwise produced by any of these methods and/or altered organisms.

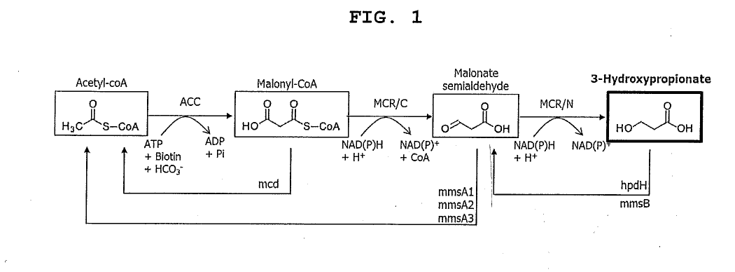

Acetyl-CoA from central metabolism is converted into malonyl-CoA by acetyl-CoA carboxylases (ACC) which can be directed into the fatty acid biosynthesis. In the presence of a malonyl-CoA reductase (MCR), malonyl-CoA can be reduced to 3-HP in a two-step reaction with the first step comprising reduction of malonyl-CoA by the MCR/C-terminal into malonate semialdehyde and conversion of the semialdehyde by the MCR/N-terminal into 3-HP. See FIG. 1. The inventors herein believe that the carbon flux through the pyruvate/acetyl-Coenzyme A node in an organism can be redirected to produce 3-HP and that organisms altered to express MCR can be used in accordance with the present invention in methods for biosynthesizing higher levels of 3-HP, derivatives thereof, and compounds related thereto.

For purposes of the present invention, by “3-hydroxypropanoic acid (3-HP)” it is meant to encompass 3-hydroxypropanate, 3-HP CoA and other C2, C3 and C4 acids and their derivatives.

For purposes of the present invention, by “derivatives and compounds related thereto” it is meant to encompass compounds derived from the same substrates and/or enzymatic reactions as compounds involved in 3-HP metabolism, byproducts of these enzymatic reactions and compounds with similar chemical structure including, but not limited to, structural analogs wherein one or more substituents of compounds involved in 3-HP metabolism are replaced with alternative substituents e.g. 02 and C3 acids and their derivatives.

For purposes of the present invention, by “higher levels of 3-HP” it is meant that the altered organisms and methods of the present invention are capable of producing increased levels of 3-HP and derivatives and compounds related thereto as compared to the same organism without alteration.

For compounds containing carboxylic acid groups such as organic monoacids, hydroxyacids, amino acids and dicarboxylic acids, these compounds may be formed or converted to their ionic salt form when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate and/or bicarbonate, sodium hydroxide, ammonia and the like. The salt can be isolated as is from the system as the salt or converted to the free acid by reducing the pH to, for example, below the lowest pKa through addition of acid or treatment with an acidic ion exchange resin.

For compounds containing amine groups such as, but not limited to, organic amines, amino acids and diamine, these compounds may be formed or converted to their ionic salt form by addition of an acidic proton to the amine to form the ammonium salt, formed with inorganic acids such as hydrochloric acid, hydrobromic acid, sulfuric acid, nitric acid, phosphoric acid, and the like; or formed with organic acids such as carbonic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid or muconic acid, and the like. The salt can be isolated as is from the system as a salt or converted to the free amine by raising the pH to, for example, above the highest pKa through addition of base or treatment with a basic ion exchange resin. Acceptable inorganic bases are known in the art and include aluminum hydroxide, calcium hydroxide, potassium hydroxide, sodium carbonate or bicarbonate, sodium hydroxide, and the like.

For compounds containing both amine groups and carboxylic acid groups such as, but not limited to, amino acids, these compounds may be formed or converted to their ionic salt form by either 1) acid addition salts, formed with inorganic acids such as hydrochloric acid, hydrobromic acid, sulfuric acid, nitric acid, phosphoric acid, and the like; or formed with organic acids such as carbonic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Acceptable inorganic bases include aluminum hydroxide, calcium hydroxide, potassium hydroxide, sodium carbonate and/or bicarbonate, sodium hydroxide, and the like, or 2) when an acidic proton present in the parent compound either is replaced by a metal ion, e.g., an alkali metal ion, an alkaline earth ion, or an aluminum ion; or coordinates with an organic base. Acceptable organic bases are known in the art and include ethanolamine, diethanolamine, triethanolamine, trimethylamine, N-methylglucamine, and the like. Acceptable inorganic bases are known in the art and include aluminum hydroxide, calcium hydroxide, potassium hydroxide, sodium carbonate, sodium hydroxide, ammonia and the like. The salt can be isolated as is from the system or converted to the free acid by reducing the pH to, for example, below the pKa through addition of acid or treatment with an acidic ion exchange resin. In one or more aspects of the invention, it is understood that the amino acid salt can be isolated as: i. at low pH, as the ammonium (salt)-free acid form; ii. at high pH, as the amine-carboxylic acid salt form; and/or iii. at neutral or midrange pH, as the free-amine acid form or zwitterion form.

In the process for biosynthesis of 3-HP and derivatives and compounds related thereto of the present invention, an organism capable of producing 3-HP and derivatives and compounds related thereto is obtained. The organism is then altered to produce more 3-HP and derivatives and compounds related thereto in the altered organism as compared to the unaltered organism.

In one nonlimiting embodiment, the organism is Cupriavidus necator (C. necator) or an organism with properties similar thereto. A nonlimiting embodiment of the organism is set for at lgcstandards-atcc with the extension .org/products/a11/17699.aspx?geo_country=gb#generalinformation of the world wide web.

C. necator (previously called Hydrogenomonas eutrophus, Alcaligenes eutropha, Ralstonia eutropha, and Wautersia eutropha) is a Gram-negative, flagellated soil bacterium of the Betaproteobacteria class. This hydrogen-oxidizing bacterium is capable of growing at the interface of anaerobic and aerobic environments and easily adapts between heterotrophic and autotrophic lifestyles. Sources of energy for the bacterium include both organic compounds and hydrogen. C. necator does not naturally contain genes for MCR and therefore does not express this enzyme. Additional properties of C. necator include microaerophilicity, copper resistance (Makar, N. S. & Casida, L. E. Int. J. of Systematic Bacteriology 1987 37(4): 323-326), bacterial predation (Byrd et al. Can J Microbiol 1985 31:1157-1163; Siliman, C. E. & Casida, L. E. Can J Microbiol 1986 32:760-762; Zeph, L. E. & Casida, L. E. Applied and Environmental Microbiology 1986 52(4):819-823) and polyhydroxybutyrate (PHB) synthesis. In addition, the cells have been reported to be capable of both aerobic and nitrate dependent anaerobic growth. A nonlimiting example of a C. necator organism useful in the present invention is a C. necator of the H16 strain. In one nonlimiting embodiment, a C. necator host of the H16 strain with at least a portion of the phaCAB gene locus knocked out (ΔphaCAB) is used.

In another nonlimiting embodiment, the organism altered in the process of the present invention has one or more of the above-mentioned properties of Cupriavidus necator.

In another nonlimiting embodiment, the organism is selected from members of the genera Ralstonia, Wautersia, Cupriavidus, Alcaligenes, Burkholderia or Pandoraea.

For the process of the present invention, the organism is altered to express malonyl-CoA reductase (MCR). In one nonlimiting embodiment, the MCR comprises Chloroflexus aurantiacus MCR (SEQ ID NO:1) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 1 or a functional fragment thereof. In one nonlimiting embodiment, the MCR is encoded by a nucleic acid sequence comprising Chloroflexus aurantiacus MCR (SEQ ID NO:2) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 2 or a functional fragment thereof. In one nonlimiting embodiment, the MCR is EC 1.2.1.75.

In one nonlimiting embodiment, the nucleic acid sequence or sequences are codon optimized for C. necator.

In one nonlimiting embodiment, the organism is further altered to redirect the carbon flux to 3-HP via interference with any one or more of a malonate semialdehyde dehydrogenase such as MMSA1, MMSA2 and/or MMSA3, enzymes that potentially degrade malonate semialdehyde into acetyl-CoA; and/or a malonyl-CoA decarboxylase (MCD) that converts malonyl-CoA back into acetyl-CoA; and/or a 3-hydroxypropionate dehydrogenase (HPDH) that converts 3-HP into malonate semialdehyde; and/or another a 3-hydroxyisobutyrate dehydrogenase (MMSB) that could putatively convert malonate semialdehyde into (S)3-hydroxybutyrate; and/or a 2-hydroxy-3-oxopropionate reductase; and/or a NAD-dependent beta-hydroxyacid dehydrogenase (mmsB), a choline dehydrogenase, a glucose-methanol-choline oxidoreductase and/or a oxidoreductase (hpdH) which converts 3-hydroxypropionate to malonate semialdehyde; and/or a CoA transferase or a CoA ligase which converts 3-hydroxypropionate to 3-hydroxypropionate-CoA; and/or one or more enzymes converting 3-hydroxypropionate to succinyl-CoA as depicted, for example, in FIG. 1. As will be understood by the skilled artisan upon reading this disclosure, one or more of any of these enzymes and/or one or more enzymes in each of these enzyme classes may be interfered with in accordance with this invention.

As used herein, by “interference with” or “interfered with” it is meant to encompass any physical or chemical change to the organism which ultimately decreases activity of the enzyme. Examples include, but are in no way limited to, mutation or deletion of a gene encoding the enzyme, addition of an enzyme inhibitor and addition of an agent which decreases or inhibits expression of the enzyme.

In one nonlimiting embodiment, the organism is further modified to eliminate phaCAB, involved in PHBs production and/or H16-A0006-9 encoding endonucleases thereby improving transformation efficiency as described in U.S. patent application Ser. No. 15/717,216, teachings of which are incorporated herein by reference.

In the process of the present invention, the altered organism is then subjected to conditions wherein 3-HP and derivatives and compounds related thereto are produced.

In the process described herein, a fermentation strategy can be used that entails anaerobic, micro-aerobic or aerobic cultivation. A fermentation strategy can entail nutrient limitation such as nitrogen, phosphate or oxygen limitation.

Under conditions of nutrient limitation a phenomenon known as overflow metabolism (also known as energy spilling, uncoupling or spillage) occurs in many bacteria (Russell, 2007). In growth conditions in which there is a relative excess of carbon source and other nutrients (e.g. phosphorous, nitrogen and/or oxygen) are limiting cell growth, overflow metabolism results in the use of this excess energy (or carbon), not for biomass formation but for the excretion of metabolites, typically organic acids. In Cupriavidus necator a modified form of overflow metabolism occurs in which excess carbon is sunk intracellularly into the storage carbohydrate polyhydroxybutyrate (PHB). In strains of C. necator which are deficient in PHB synthesis this overflow metabolism can result in the production of extracellular overflow metabolites. The range of metabolites that have been detected in PHB deficient C. necator strains include acetate, acetone, butanoate, cis-aconitate, citrate, ethanol, fumarate, 3-hydroxybutanoate, propan-2-ol, malate, methanol, 2-methyl-propanoate, 2-methyl-butanoate, 3-methyl-butanoate, 2-oxoglutarate, meso-2,3-butanediol, acetoin, DL-2,3-butanediol, 2-methylpropan-1-ol, propan-1-ol, lactate 2-oxo-3-methylbutanoate, 2-oxo-3-methylpentanoate, propanoate, succinate, formic acid and pyruvate. The range of overflow metabolites produced in a particular fermentation can depend upon the limitation applied (e.g. nitrogen, phosphate, oxygen), the extent of the limitation, and the carbon source provided (Schlegel, H. G. & Vollbrecht, D. Journal of General Microbiology 1980 117:475-481; Steinbüchel, A. & Schlegel, H. G. Appl Microbiol Biotechnol 1989 31: 168; Vollbrecht et al. Eur J Appl Microbiol Biotechnol 1978 6:145-155; Vollbrecht et al. European J. Appl. Microbiol. Biotechnol. 1979 7: 267; Vollbrecht, D. & Schlegel, H. G. European J. Appl. Microbiol. Biotechnol. 1978 6: 157; Vollbrecht, D. & Schlegel, H. G. European J. Appl. Microbiol. Biotechnol. 1979 7: 259).

Applying a suitable nutrient limitation in defined fermentation conditions can thus result in an increase in the flux through a particular metabolic node. The application of this knowledge to C. necator strains genetically modified to produce desired chemical products via the same metabolic node can result in increased production of the desired product.

A cell retention strategy using a ceramic hollow fiber membrane can be employed to achieve and maintain a high cell density during fermentation. The principal carbon source fed to the fermentation can derive from a biological or non-biological feedstock. The biological feedstock can be, or can derive from, monosaccharides, disaccharides, lignocellulose, hemicellulose, cellulose, paper-pulp waste, black liquor, lignin, levulinic acid and formic acid, triglycerides, glycerol, fatty acids, agricultural waste, thin stillage, condensed distillers' solubles or municipal waste such as fruit peel/pulp. The non-biological feedstock can be, or can derive from, natural gas, syngas, CO₂/H₂, CO, H₂, O₂, methanol, ethanol, non-volatile residue (NVR) a caustic wash waste stream from cyclohexane oxidation processes or waste stream from a chemical industry such as, but not limited to a carbon black industry or a hydrogen-refining industry, or petrochemical industry, a nonlimiting example being a PTA-waste stream.

In one nonlimiting embodiment, at least one of the enzymatic conversions of the 3-HP production method comprises gas fermentation within the altered Cupriavidus necator host, or a member of the genera Ralstonia, Wautersia, Alcaligenes, Burkholderia and Pandoraea, and other organism having one or more of the above-mentioned properties of Cupriavidus necator. In this embodiment, the gas fermentation may comprise at least one of natural gas, syngas, CO, H₂, O₂, CO₂/H₂, methanol, ethanol, non-volatile residue, caustic wash from cyclohexane oxidation processes, or waste stream from a chemical industry such as, but not limited to a carbon black industry or a hydrogen-refining industry, or petrochemical industry. In one nonlimiting embodiment, the gas fermentation comprises CO₂/H₂.

The methods of the present invention may further comprise recovering produced 3-HP or derivatives or compounds related thereto. Once produced, any method can be used to isolate the 3-HP or derivatives or compounds related thereto.

The present invention also provides altered organisms capable of biosynthesizing increased amounts of 3-HP and derivatives and compounds related thereto as compared to the unaltered organism. In one nonlimiting embodiment, the altered organism of the present invention is a genetically engineered strain of Cupriavidus necator capable of producing 3-HP and derivatives and compounds related thereto. In another nonlimiting embodiment, the organism to be altered is selected from members of the genera Ralstonia, Wautersia, Alcaligenes, Cupriavidus, Burkholderia and Pandoraea, and other organisms having one or more of the above-mentioned properties of Cupriavidus necator. In one nonlimiting embodiment, the present invention relates to a substantially pure culture of the altered organism capable of producing 3-HP and derivatives and compounds related thereto via a MCR pathway.

As used herein, a “substantially pure culture” of an altered organism is a culture of that microorganism in which less than about 40% (i.e., less than about 35%; 30%; 25%; 20%; 15%; 10%; 5%; 2%; 1%; 0.5%; 0.25%; 0.1%; 0.01%; 0.001%; 0.0001%; or even less) of the total number of viable cells in the culture are viable cells other than the altered microorganism, e.g., bacterial, fungal (including yeast), mycoplasmal, or protozoan cells. The term “about” in this context means that the relevant percentage can be 15% of the specified percentage above or below the specified percentage. Thus, for example, about 20% can be 17% to 23%. Such a culture of altered microorganisms includes the cells and a growth, storage, or transport medium. Media can be liquid, semi-solid (e.g., gelatinous media), or frozen. The culture includes the cells growing in the liquid or in/on the semi-solid medium or being stored or transported in a storage or transport medium, including a frozen storage or transport medium. The cultures are in a culture vessel or storage vessel or substrate (e.g., a culture dish, flask, or tube or a storage vial or tube).

Altered organisms of the present invention comprise at least one genome-integrated synthetic operon encoding an enzyme.

In one nonlimiting embodiment, the altered organism is produced by integration of a synthetic operon encoding MCR into the host genome.

In one nonlimiting embodiment, the MCR comprises Chloroflexus aurantiacus MCR (SEQ ID NO:1) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 1 or a functional fragment thereof. In one nonlimiting embodiment, the MCR is encoded by a nucleic acid sequence comprising Chloroflexus aurantiacus MCR (SEQ ID NO:2) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 2 or a functional fragment thereof. In one nonlimiting embodiment, the MCR is EC 1.2.1.75.

In one nonlimiting embodiment, the nucleic acid sequence or sequences are codon optimized for C. necator.

In one nonlimiting embodiment, the organism is further altered to redirect the carbon flux to 3-HP via interference with any one or more of a malonate semialdehyde dehydrogenase such as MMSA1, MMSA2 and/or MMSA3, enzymes that potentially degrade malonate semialdehyde into acetyl-CoA; and/or a malonyl-CoA decarboxylase (MCD) that converts malonyl-CoA back into acetyl-CoA; and/or a 3-hydroxypropionate dehydrogenase (HPDH) that converts 3-HP into malonate semialdehyde; and/or another a 3-hydroxyisobutyrate dehydrogenase (MMSB) that could putatively convert malonate semialdehyde into (S)3-hydroxybutyrate; and/or a 2-hydroxy-3-oxopropionate reductase; and/or a NAD-dependent beta-hydroxyacid dehydrogenase (mmsB), a choline dehydrogenase, a glucose-methanol-choline oxidoreductase and/or a oxidoreductase (hpdH) which converts 3-hydroxypropionate to malonate semialdehyde; and/or a CoA transferase or a CoA ligase which converts 3-hydroxypropionate to 3-hydroxypropionate-CoA; and/or one or more enzymes converting 3-hydroxypropionate to succinyl-CoA as depicted, for example, in FIG. 1.

In one nonlimiting embodiment, the organism is further modified to eliminate phaCAB, involved in PHBs production and/or H16-A0006-9 encoding endonucleases thereby improving transformation efficiency.

Alternative pathways to 3-HP for use in the processes and organisms encompassed by the present invention include, but are not limited to pathways comprising a malonyl-CoA reductase such as from Sulfobolus tokodaii. Such altered organisms for use in the processes of the present invention may further comprise a 3-hydroxypropionate dehydrogenase such as from Metallosphaera sedula or a 3-hydroxyisobutyrate dehydrogenase such as from P. aeruginosa as described by Chen et al. (Metabolic Engineering 2014 22:104-109) for Pseudomonas cerevisiae.

The percent identity (and/or homology) between two amino acid sequences as disclosed herein can be determined as follows. First, the amino acid sequences are aligned using the BLAST 2 Sequences (Bl2seq) program from the stand-alone version of BLAST containing BLASTP version 2.0.14. This stand-alone version of BLAST can be obtained from the U.S. government's National Center for Biotechnology. Information web site (www with the extension ncbi.nlm.nih.gov). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq c:\seq1.txt-j c:\seq2.txt -p blastp-o c:\output.txt. If the two compared sequences share homology (identity), then the designated output file will present those regions of homology as aligned sequences. If the two compared sequences do not share homology (identity), then the designated output file will not present aligned sequences. Similar procedures can be followed for nucleic acid sequences except that blastn is used.

Once aligned, the number of matches is determined by counting the number of positions where an identical amino acid residue is presented in both sequences. The percent identity (homology) is determined by dividing the number of matches by the length of the full-length polypeptide amino acid sequence followed by multiplying the resulting value by 100. It is noted that the percent identity (homology) value is rounded to the nearest tenth. For example, 90.11, 90.12, 90.13, and 90.14 is rounded down to 90.1, while 90.15, 90.16, 90.17, 90.18, and 90.19 is rounded up to 90.2. It also is noted that the length value will always be an integer.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) is obtained, using appropriate codon bias tables for that species.

Functional fragments of any of the polypeptides or nucleic acid sequences described herein can also be used in the methods and organisms disclosed herein. The term “functional fragment” as used herein refers to a peptide or fragment of a polypeptide or a nucleic acid sequence fragment encoding a peptide fragment of a polypeptide that has at least about 25% (e.g., at least: 30%; 40%; 50%; 60%; 70%; 75%; 80%; 85%; 90%; 95%; 98%; 99%; 100%; or even greater than 100%) of the activity of the corresponding mature, full-length, polypeptide. The functional fragment can generally, but not always, be comprised of a continuous region of the polypeptide, wherein the region has functional activity.

Functional fragments may range in length from about 10% up to 99% (inclusive of all percentages in between) of the original full-length sequence.

This document also provides (i) functional variants of the enzymes used in the methods of the document and (ii) functional variants of the functional fragments described above. Functional variants of the enzymes and functional fragments can contain additions, deletions, or substitutions relative to the corresponding wild-type sequences. Enzymes with substitutions will generally have not more than 50 (e.g., not more than one, two, three, four, five, six, seven, eight, nine, ten, 12, 15, 20, 25, 30, 35, 40, or 50) amino acid substitutions (e.g., conservative substitutions). This applies to any of the enzymes described herein and functional fragments. A conservative substitution is a substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution. By contrast, a nonconservative substitution is a substitution of one amino acid for another with dissimilar characteristics.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids. Additions (addition variants) include fusion proteins containing: (a) any of the enzymes described herein or a fragment thereof; and (b) internal or terminal (C or N) irrelevant or heterologous amino acid sequences. In the context of such fusion proteins, the term “heterologous amino acid sequences” refers to an amino acid sequence other than (a). A heterologous sequence can be, for example a sequence used for purification of the recombinant protein (e.g., FLAG, polyhistidine (e.g., hexahistidine), hemagluttanin (HA), glutathione-S-transferase (GST), or maltose binding protein (MBP)). Heterologous sequences also can be proteins useful as detectable markers, for example, luciferase, green fluorescent protein (GFP), or chloramphenicol acetyl transferase (CAT). In some embodiments, the fusion protein contains a signal sequence from another protein. In certain host cells (e.g., yeast host cells), expression and/or secretion of the target protein can be increased through use of a heterologous signal sequence. In some embodiments, the fusion protein can contain a carrier (e.g., KLH) useful, e.g., in eliciting an immune response for antibody generation) or ER or Golgi apparatus retention signals. Heterologous sequences can be of varying length and in some cases can be a longer sequences than the full-length target proteins to which the heterologous sequences are attached.

Endogenous genes of the organisms altered for use in the present invention also can be disrupted to prevent the formation of undesirable metabolites or prevent the loss of intermediates in the pathway through other enzymes acting on such intermediates. In one nonlimiting embodiment, the organism is further altered to redirect the carbon flux to 3-HP via interference with any one or more of a malonate semialdehyde dehydrogenase such as MMSA1, MMSA2 and/or MMSA3, enzymes that potentially degrade malonate semialdehyde into acetyl-CoA; and/or a malonyl-CoA decarboxylase (MCD) that converts malonyl-CoA back into acetyl-CoA; and/or a 3-hydroxypropionate dehydrogenase (HPDH) that converts 3-HP into malonate semialdehyde; and/or another a 3-hydroxyisobutyrate dehydrogenase (MMSB) that could putatively convert malonate semialdehyde into (S)3-hydroxybutyrate; and/or a 2-hydroxy-3-oxopropionate reductase; and/or a NAD-dependent beta-hydroxyacid dehydrogenase (mmsB), a choline dehydrogenase, a glucose-methanol-choline oxidoreductase and/or a oxidoreductase (hpdH) which converts 3-hydroxypropionate to malonate semialdehyde; and/or a CoA transferase or a CoA ligase which converts 3-hydroxypropionate to 3-hydroxypropionate-CoA; and/or one or more enzymes converting 3-hydroxypropionate to succinyl-CoA as depicted, for example, in FIG. 1. In one nonlimiting embodiment, the organism is further modified to eliminate phaCAB, involved in PHBs production and/or H16-A0006-9 encoding endonucleases thereby improving transformation efficiency.

Thus, as described herein, altered organisms can include exogenous nucleic acids encoding MCR, as described herein, as well as modifications to endogenous genes.

The term “exogenous” as used herein with reference to a nucleic acid (or a protein) and an organism refers to a nucleic acid that does not occur in (and cannot be obtained from) a cell of that particular type as it is found in nature or a protein encoded by such a nucleic acid. Thus, a non-naturally-occurring nucleic acid is considered to be exogenous to a host once in the host. It is important to note that non-naturally-occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a host cell once introduced into the host, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be exogenous to a particular host microorganism. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) (or a protein) and a host refers to a nucleic acid (or protein) that does occur in (and can be obtained from) that particular host as it is found in nature. Moreover, a cell “endogenously expressing” a nucleic acid (or protein) expresses that nucleic acid (or protein) as does a host of the same particular type as it is found in nature. Moreover, a host “endogenously producing” or that “endogenously produces” a nucleic acid, protein, or other compound produces that nucleic acid, protein, or compound as does a host of the same particular type as it is found in nature.

The present invention also provides exogenous genetic molecules of the nonnaturally occurring organisms disclosed herein such as, but not limited to, codon optimized nucleic acid sequences, expression constructs and/or synthetic operons.

In one nonlimiting embodiment, the exogenous genetic molecule comprises a codon optimized nucleic acid sequence optimized for C. necator. In one nonlimiting embodiment, the exogenous genetic molecule comprises a nucleic acid sequence comprising Chloroflexus aurantiacus MCR (SEQ ID NO:2) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 2 or a functional fragment thereof.

In another nonlimiting embodiment, the exogenous genetic molecule comprises an MCR expression construct.

In another nonlimiting embodiment, the exogenous genetic molecule comprises a synthetic operon encoding MCR.

The utility of MCR in increasing biosynthesis of 3-HP was evaluated in a genetically engineered strain of Cupriavidus necator.

Malonyl-Coenzyme A Reductase (MCR) from Chloroflexus aurantiacus was cloned into a pBBR1-based plasmid and transformed into Cupriavidus necator H16 ΔphaCAB:ΔA0006-9: ΔmmsA1:Δmcd:ΔmmsA2-hpdH:ΔmmsA3-mmsB. An equivalent empty vector (with the promoter present, pBAD) was used as a negative control. The accumulation of 3-HP was assessed in a shake flask experiment after a 24-hour period and 3-HP was detected by LCMS.

Also provided by the present invention are 3-HP and derivatives and compounds related thereto bioderived from an altered organism according to any of methods described herein.

Further, the present invention relates to means and processes for use of these means for biosynthesis of 3-HP including derivatives thereof and/or compounds related thereto. Nonlimiting examples of such means include altered organisms and exogenous genetic molecules as described herein as well as any of the molecules as depicted in FIG. 1.

In addition, the present invention provides bio-derived, bio-based, or fermentation-derived products produced using the methods and/or altered organisms disclosed herein. In one nonlimiting embodiment, a bio-derived, bio-based or fermentation derived product is produced in accordance with the exemplary central metabolism depicted in FIG. 1. Examples of such products include, but are not limited to, compositions comprising at least one bio-derived, bio-based, or fermentation-derived compound or any combination thereof, as well as polymers, plastics, molded substances, formulations and semi-solid or non-semi-solid streams comprising one or more of the bio-derived, bio-based, or fermentation-derived compounds or compositions, combinations or products thereof.

The following section provides further illustration of the methods and materials of the present invention. These Examples are illustrative only and are not intended to limit the scope of the invention in any way.

### Examples

### Constructs and Strains

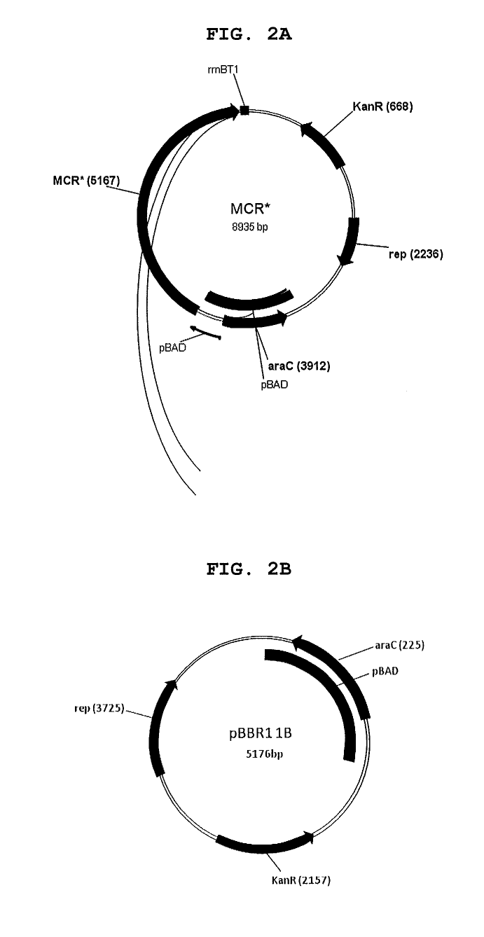

Malonyl-Coenzyme A Reductase (MCR) from Chloroflexus aurantiacus (Ac no. AAS20429) was cloned into a pBBR1-based plasmid using standard cloning techniques. The expression vector and the correspondent empty vector (MCR* and pBBR1_1B; maps in FIG. 2) were transformed in the Cupriavidus necator H16 ΔphaCAB:ΔA0006-9: ΔmmsA1:Δmcd:ΔmmsA2-hpdH:ΔmmsA3-mmsB by electroporation. The recipient strain contains 6 genetic loci knocked out: 1) ΔphaCAB, involved in PHBs production; 2) ΔA0006-9, encoding endonucleases which improves transformation efficiency; 3) ΔmmsA1, which encodes a malonate semialdehyde dehydrogenase, an enzyme that potentially converts malonate semialdehyde into acetyl-CoA; 4) Δmcd, encoding a malonyl-CoA decarboxylase that converts malonyl-CoA back into acetyl-CoA; 5) ΔmmsA2-hpdH, encoding another malonate semialdehyde dehydrogenase that converts malonate semialdehyde into acetyl-CoA and a 3-hydroxypropionate dehydrogenase that converts 3-HP into malonate semialdehyde; 6) ΔmmsA3-mmsB, encoding another malonate semialdehyde dehydrogenase that converts malonate semialdehyde into acetyl-CoA and a 3-hydroxyisobutyrate dehydrogenase that that converts 3-HP into malonate semialdehyde.

Examples of hpdH and mmsB Enzymes for Interference

Nonlimiting examples of 3-hydroxyisobutyrate dehydrogenase, 2-hydroxy-3-oxopropionate reductase and NAD-dependent beta-hydroxyacid dehydrogenase referred to collectively as mmsB, and choline dehydrogenase, glucose-methanol-choline oxidoreductase and oxidoreductase referred to collectively as hpdH, which converts 3-hydroxypropionate to malonate semialdehyde are disclosed in Table 1. Experiments have been conducted where H16 A3663 and/or H16-B1190 of C. necator have been deleted. However, as will be understood by the skilled artisan upon reading this disclosure, more than one of these enzymes may be interfered with in accordance with this invention.

TABLE 1

| | | % Identity (>30%), covering >90% | |
|---|---|---|---|
| | | of sequence | |
| | Enzyme/ | C. necator | P. denitrificans |
| hpdH | Accession No. | H16_A3663 | YP_007659112 |
| H16_A3663 | choline | 100 | 60 |
| | dehydrogenase | | |
| | WP_010811289.1 | | |
| H16_B1851 | glucose-methanol- | 46 | 46 |
| | choline | | |
| | oxidoreductase | | |
| | WP_010810328.1 | | |
| H16_B1532 | Oxidoreductase | 43 | 42 |
| | WP_011617294.1 | | |
| H16_B2131 | choline | 41 | 41 |
| | dehydrogenase | | |
| | WP_010811005.1 | | |
| H16_A0233 | choline | 39 | 41 |
| | dehydrogenase | | |
| | WP_011614415.1 | | |
| H16_B0411 | choline | 39 | 41 |
| | dehydrogenase and | | |
| | alkyl sulfatase | | |
| | WP_011616571.1 | | |

| | | % Identity (>30%), covering >90% of sequence | | |
|---|---|---|---|---|
| | Enzyme/ | C. necator | P. denitrificans | P. denitrificans |
| mmsB | Accession No. | H16_B1190 | YP_007656737 | YP_007658098 |
| H16_B1190 | 3-hydroxyisobutyrate | 100 | 52 | 66 |
| | dehydrogenase | | | |
| | WP_011617070.1 | | | |
| H16_B1750 | 3-hydroxyisobutyrate | 45 | 44 | 46 |
| | dehydrogenase | | | |
| | WP_011617453.1 | | | |
| H16_A3004 | 3-hydroxyisobutyrate | 38 | 37 | 38 |
| | dehydrogenase | | | |
| | WP_010814951.1 | | | |
| H16_B1657 | 3-hydroxyisobutyrate | 34 | 33 | |
| | dehydrogenase | | | |
| | WP_011617380.1 | | | |
| H16_A3600 | 2-hydroxy-3- | 35 | 33 | 35 |
| | oxopropionate | | | |
| | reductase | | | |
| | WP_010812149.1 | | | |
| H16_B0941 | NAD-dependent beta- | 36 | 35 | 39 |
| | hydroxyacid | | | |
| | dehydrogenase | | | |
| | WP_010809660.1 | | | |
| H16_A1562 | 3-hydroxyisobutyrate | 31 | 31 | 37 |
| | dehydrogenase | | | |
| | WP_011615152.1 | | | |
| H16_A1239 | 3-hydroxyisobutyrate | 30 | | |
| | dehydrogenase | | | |
| | WP_011614949.1 | | | |

*by % Identity (>30%), covering >90% of sequence it is meant that the genes all have at least 30% sequence identity along at least any 90% of the length, relative to the first C. necator gene listed which has already been knocked out.

### Examples of CoA Transferase or Ligase Enzymes for Interference

Nonlimiting examples of CoA transferase or ligase enzymes which convert 3-hydroxypropionate to 3-hydroxypropionate-CoA are disclosed in SEQ ID NOs: 4 through 19. See Fukui et al. Biomacromolecules 2009 13 10(4):700-6 and Volodina et al. Appl Microbiol Biotechnol. 2014 98(8): 3579-89. As will be understood by the skilled artisan upon reading this disclosure, more than one of these enzymes may be interfered with in accordance with this invention.

### 3-HP Bioassay and Analytical Detection

To assess 3-HP accumulation in the strains generated, strains were pre-cultured overnight (at least 2 biological replicates). The bioassay was initiated at OD₆₀₀=0.4-0.5 in media supplemented with appropriate antibiotic. Cultures were incubated at 30° C., 230 rpm for 8 hours (OD₆₀₀=˜0.5-0.7) and induced with 0.3% of L-arabinose. After 24 hours incubation at 30° C. and 230 rpm, cultures were spun down and 3-HP was detected in the supernatant (3 technical replicas) by LCMS.

TABLE 2
Sequence Information for Sequences in Sequence Listing

| SEQ | |
|---|---|
| ID NO: | Sequence Description |
| 1 | Amino acid sequence of Ca MCR |
| 2 | Nucleic acid sequence of Ca MCR |
| 3 | Nucleic acid sequence of pBBR1 (1B): pBAD |
| 4 | Amino Acid Sequence for Propionate CoA-transferase (pct); |
| | EC 2.8.3.1; H16 A2718; CAJ93797 |
| 5 | Nucleic acid sequence for Propionate CoA-transferase (pct); |
| | EC 2.8.3.1; H16 A2718; CAJ93797 |
| 6 | Amino acid sequence for Propionate-CoA ligase (prpE); |
| | EC 6.2.1.17 H16 A2462; CAJ93551 |
| 7 | Nucleic acid sequence for Propionate-CoA ligase (prpE); |
| | EC 6.2.1.17 H16 A2462; CAJ93551 |
| 8 | Amino acid sequence for Acetyl-CoA synthetase/ligase; |
| | EC 6.2.1.1 H16 A1197; CAJ92338 |
| 9 | Nucleic acid sequence for Acetyl-CoA synthetase/ligase; |
| | EC 6.2.1.1 H16 A1197; CAJ92338 |
| 10 | Amino Acid sequence for EC 6.2.1.1 H16_A1616; CAJ92748 |
| 11 | Nucleic acid sequence for EC 6.2.1.1 H16_A1616; CAJ92748 |
| 12 | Amino acid sequence for EC 6.2.1.1 H16_A2525; CAJ93612 |
| 13 | Nucleic acid sequence for EC 6.2.1.1 H16_A2525; CAJ93612 |
| 14 | Amino Acid sequence for EC 6.2.1.1 H16_B0396; CAJ95185 |
| 15 | Nucleic acid sequence for EC 6.2.1.1 H16_B0396; CAJ95185 |
| 16 | Amino Acid sequence for EC 6.2.1.1 H16_B0834; CAJ95626 |
| 17 | Nucleic acid sequence for EC 6.2.1.1 H16_B0834; CAJ95626 |
| 18 | Amino Acid sequence for EC 6.2.1.1 H16_B1102; CAJ95893 |
| 19 | Nucleic acid sequence for EC 6.2.1.1 H16_B1102; CAJ95893 |

1. A process for biosynthesis of 3-hydroxypropanoic acid (3-HP), derivatives thereof and/or compounds related thereto, said process comprising:
obtaining an organism capable of producing 3-HP, derivatives thereof and/or compounds related thereto;
altering the organism; and
producing more 3-HP, derivatives thereof and/or compounds related thereto by the altered organism as compared to the unaltered organism.

2. The process of claim 1 wherein the organism is C. necator or an organism with properties similar thereto.

3. The process of claim 1 wherein the organism is altered to express malonyl-CoA reductase (MCR).

4. The process of claim 3 wherein the MCR is from C. aurantiacus or S. tokodaii.

5. The process of claim 3 wherein the MCR comprises Chloroflexus aurantiacus MCR (SEQ ID NO:1) or a polypeptide with similar enzymatic activities exhibiting at least about about 50% sequence identity to an amino acid sequence set forth in SEQ ID NO: 1 or a functional fragment thereof or is encoded by a nucleic acid sequence comprising Chloroflexus aurantiacus MCR (SEQ ID NO:2) or a nucleic acid sequence encoding a polypeptide with similar enzymatic activities exhibiting at least about 50% sequence identity to the nucleic acid sequence set forth in SEQ ID NO: 2 or a functional fragment thereof.

6. (canceled)

7. The process of claim 1 wherein the organism is further altered to redirect carbon flux to 3-HP via interference with one or more of a malonate semialdehyde dehydrogenase, a malonyl-CoA decarboxylase, a 3-hydroxypropionate dehydrogenase, a 2-hydroxy-3-oxopropionate reductase, a NAD-dependent beta-hydroxyacid dehydrogenase, a choline dehydrogenase, a glucose-methanol-choline oxidoreductase, an oxidoreductase, a CoA transferase or a CoA ligase and/or one or more enzymes converting 3-hydroxypropionate to succinyl-CoA.

8. The process of claim 1 wherein the organism is further altered to eliminate phaCAB, involved in PHBs production and/or H16-A0006-9 encoding endonucleases thereby improving transformation efficiency.

9. The process of claim 5 wherein the nucleic acid sequence is codon optimized for C. necator.

10. An altered organism capable of producing and/or accumulating more 3-HP, derivatives thereof and/or compounds related thereto as compared to an unaltered organism.

11. The altered organism of claim 10 which is C. necator or an organism with properties similar thereto.

12. The altered organism of claim 10 which expresses MCR.

13. The altered organism of claim 12 wherein the MCR is from C. aurantiacus or S. tokodaii.

14. The altered organism of claim 12 wherein the MCR comprises Chloroflexus aurantiacus MCR (SEQ ID NO:1) or a polypeptide with similar enzymatic activities exhibiting at least about 50% sequence identity to an amino acid sequence set forth in SEQ ID NO: 1 or a functional fragment thereof or is encoded by a nucleic acid sequence comprising Chloroflexus aurantiacus MCR (SEQ ID NO:2) or a nucleic acid sequence encoding a polypeptide with similar enzymatic activities exhibiting at least about 50% sequence identity to the nucleic acid sequence set forth in SEQ ID NO: 2 or a functional fragment thereof.

15. (canceled)

16. The altered organism of claim 10 wherein the organism is further altered to redirect carbon flux to 3-HP via interference with one or more of a malonate semialdehyde dehydrogenase, a malonyl-CoA decarboxylase, a 3-hydroxypropionate dehydrogenase, a 2-hydroxy-3-oxopropionate reductase, a NAD-dependent beta-hydroxyacid dehydrogenase, a choline dehydrogenase, a glucose-methanol-choline oxidoreductase, an oxidoreductase, a CoA transferase or a CoA ligase and/or one or more enzymes converting 3-hydroxypropionate to succinyl-CoA.

17. The altered organism of claim 10 wherein the organism is further altered to eliminate phaCAB, involved in PHBs production and/or H16-A0006-9 encoding endonucleases thereby improving transformation efficiency.

18. The altered organism of claim 14 wherein the nucleic acid sequence is codon optimized for C. necator.

19. A bio-derived, bio-based, or fermentation-derived product produced from the method of claim 1, wherein said product comprises:
(i) a composition comprising at least one bio-derived, bio-based, or fermentation-derived compound or any combination thereof;
(ii) a bio-derived, bio-based, or fermentation-derived polymer comprising the bio-derived, bio-based, or fermentation-derived composition or compound of (i), or any combination thereof;
(iii) a bio-derived, bio-based, or fermentation-derived plastic comprising the bio-derived, bio-based, or fermentation-derived compound or bio-derived, bio-based, or fermentation-derived composition of (i), or any combination thereof or the bio-derived, bio-based, or fermentation-derived polymer of (ii), or any combination thereof;
(iv) a molded substance obtained by molding the bio-derived, bio-based, or fermentation-derived polymer of (ii), or the bio-derived, bio-based, or fermentation-derived plastic of (iii), or any combination thereof;
(v) a bio-derived, bio-based, or fermentation-derived formulation comprising the bio-derived, bio-based, or fermentation-derived composition of (i), the bio-derived, bio-based, or fermentation-derived compound of (i), the bio-derived, bio-based, or fermentation-derived polymer of (ii), the bio-derived, bio-based, or fermentation-derived plastic of (iii), or the bio-derived, bio-based, or fermentation-derived molded substance of (iv), or any combination thereof; or
(vi) a bio-derived, bio-based, or fermentation-derived semi-solid or a non-semi-solid stream, comprising the bio-derived, bio-based, or fermentation-derived composition of (i), the bio-derived, bio-based, or fermentation-derived compound of (i), the bio-derived, bio-based, or fermentation-derived polymer of (ii), the bio-derived, bio-based, or fermentation-derived plastic of (iii), the bio-derived, bio-based, or fermentation-derived formulation of (iv), or the bio-derived, bio-based, or fermentation-derived molded substance of (v), or any combination thereof.

20. A bio-derived, bio-based or fermentation derived product produced in accordance with the central metabolism depicted in FIG. 1.

21. An exogenous genetic molecule of the altered organism of claim 10.

22. The exogenous genetic molecule of claim 21 comprising a codon optimized nucleic acid sequence or an expression construct or synthetic operon for MCR.

23. The exogenous genetic molecule of claim 22 codon optimized for C. necator.

24. The exogenous genetic molecule of claim 21 comprising a nucleic acid sequence comprising Chloroflexus aurantiacus MCR (SEQ ID NO:2) or a nucleic acid sequence encoding a polypeptide with similar enzymatic activities exhibiting at least about 50% sequence identity to the nucleic acid sequence set forth in SEQ ID NO: 2 or a functional fragment thereof.

25. (canceled)

26. A process for the biosynthesis of 3-HP, derivatives thereof and/or compounds related thereto, said process comprising providing a means capable of producing 3-HP, derivatives thereof and/or compounds related thereto and producing 3-HP, derivatives thereof and/or compounds related thereto with said means.

27. A process for biosynthesis of 3-HP, and derivatives thereof, and compounds related thereto, said process comprising:
a step for performing a function of altering an organism capable of producing 3-HP, derivatives thereof, and/or compounds related thereto such that the altered organism produces more 3-HP, derivatives thereof, and/or compounds compared to a corresponding unaltered organism; and
a step for performing a function of producing 3-HP, derivatives thereof, and/or compounds related thereto in the altered organism.

28-29. (canceled)


SEQUENCE LISTING (19 sequences)

SEQ ID NO: 1 - protein, 1220 aa, Chloroflexus aurantiacus
MSGTGRLAGKIALITGGAGNIGSELTRRFLAEGATVIISGRNRAKLTALAERMQAEAGVPAKRIDLEVMDGSDPVAVRAGIEAIVARHGQIDILVNNAGSAGAQRRLAEIPLTEAELGPGAEETLHASIANLLGMGWHLMRIAAPHMPVGSAVINVSTIFSRAEYYGRIPYVTPKAALNALSQLAARELGARGIRVNTIFPGPIESDRIRTVFQRMDQLKGRPEGDTAHHFLNTMRLCRANDQGALERRFPSVGDVADAAVFLASAESAALSGETIEVTHGMELPACSETSLLARTDLRTIDASGRTTLICAGDQIEEVMALTGMLRTCGSEVIIGFRSAAALAQFEQAVNESRRLAGADFTPPIALPLDPRDPATIDAVFDWGAGENTGGIHAAVILPATSHEPAPCVIEVDDERVLNFLADEITGTIVIASRLARYWQSQRLTPGARARGPRVIFLSNGADQNGNVYGRIQSAAIGQLIRVWRHEAELDYQRASAAGDHVLPPVWANQIVRFANRSLEGLEFACAWTAQLLHSQRHINEITLNIPANISATTGARSASVGWAESLIGLHLGKVALITGGSAGIGGQIGRLLALSGARVMLAARDRHKLEQMQAMIQSELAEVGYTDVEDRVHIAPGCDVSSEAQLADLVERTLSAFGTVDYLINNAGIAGVEEMVIDMPVEGWRHTLFANLISNYSLMRKLAPLMKKQGSGYILNVSSYFGGEKDAAIPYPNRADYAVSKAGQRAMAEVFARFLGPEIQINAIAPGPVEGDRLRGTGERPGLFARRARLILENKRLNELHAALIAAARTDERSMHELVELLLPNDVAALEQNPAAPTALRELARRFRSEGDPAASSSSALLNRSIAAKLLARLHNGGYVLPADIFANLPNPPDPFFTRAQIDREARKVRDGIMGMLYLQRMPTEFDVAMATVYYLADRNVSGETFHPSGGLRYERTPTGGELFGLPSPERLAELVGSTVYLIGEHLTEHLNLLARAYLERYGARQVVMIVETETGAETMRRLLHDHVEAGRLMTIVAGDQIEAAIDQAITRYGRPGPVVCTPFRPLPTVPLVGRKDSDWSTVLSEAEFAELCEHQLTHHFRVARKIALSDGASLALVTPETTATSTTEQFALANFIKTTLHAFTATIGVESERTAQRILINQVDLTRRARAEEPRDPHERQQELERFIEAVLLVTAPLPPEADTRYAGRIHRGRAITV

SEQ ID NO: 2 - DNA, 3663 bp, Chloroflexus aurantiacus
atgtccgggacgggccgcctggccggcaaaatcgccctcatcacgggcggcgccggcaacatcgggtcggagctgacccgccgctttctggccgagggcgccacggtgatcatcagtggccgtaatcgggcgaagctgaccgcgctcgcggagcgcatgcaagccgaagctggcgtcccggccaaacggatcgacctggaagtgatggatggctcggacccggtggcggtgcgcgccggcattgaggcgatcgtcgcccgccacggccagatcgatattttggtgaacaacgccgggtcggcgggggcgcagcgccggctggcggagatcccgctgacggaggcggaactgggccccggcgccgaagaaacgctgcacgcctccatcgccaacctcctgggcatgggctggcacctgatgcggatcgccgccccccacatgcctgtggggtccgccgtgatcaatgtctcgactatcttctcgcgggccgagtactatggccgcatcccgtacgtcacgcccaaagcggccctgaatgcgctgtcccaactcgccgcccgggaactgggcgcccgcggcatccgggtcaacacgatctttccgggccccatcgagtccgatcgcatccggacggtgttccaacggatggatcagctgaagggccgcccggagggcgatacggcccatcactttctgaatacgatgcggctgtgccgcgccaacgatcagggcgccctggagcggcgcttcccgagcgtcggcgacgtggcggacgccgcggtgttcctggcgtcggccgaaagcgccgccctgagcggggaaacgatcgaagtcacccacggcatggagctgcccgcgtgtagcgaaacctccctgctggcgcgcacggacctccgcacgatcgacgcgtcgggccgcacgacgctgatctgcgcgggggatcaaatcgaagaggtcatggcgctgaccggcatgctgcgaacgtgtggctcggaagtgatcatcggcttccgctcggccgcggccctggcgcaattcgaacaggccgtcaacgagtcgcgccgcctcgccggcgcggacttcaccccccccattgcactgccactggacccgcgcgacccggccaccatcgacgccgtgttcgattggggtgcgggtgagaacaccggcggcattcatgcggccgtcatcctgcctgccacgtcccacgagccggcgccgtgcgtgatcgaggtggacgatgaacgcgtcctgaacttcctcgcggacgaaatcaccgggaccatcgtgatcgcgtcgcgcctggcccgctactggcagtctcagcgcctgacgccgggggcccgcgcccggggtccgcgcgtgatctttctgagcaacggcgccgatcaaaacggcaacgtgtacggccgcattcagagcgccgccatcggccagctcatccgcgtgtggcgccacgaggcggagctggattatcagcgcgcgtccgccgccggcgaccacgtgctgccgcccgtctgggccaatcagatcgtgcgcttcgccaataggtccctggaaggcctggaatttgcgtgcgcctggacggcccagctgctgcacagccagcgccacatcaatgaaattacgctgaacatcccggccaacatctccgccacgacgggggcgcgctcggcttcggtcggatgggcggaatccctgattggcctgcatctggggaaagtggccctgattaccggcggctcggcggggatcggggggcagattggccggttactcgcgctgagcggcgctcgggtgatgctcgccgcgcgcgaccgccataaactggagcagatgcaagccatgatccagagcgagctggccgaggtgggctataccgacgtggaggaccgggtgcatatcgccccggggtgcgatgtgagcagcgaggcgcagctggctgacctcgtcgagcgcaccctgtccgcctttggcacggtggactacctgatcaataacgccggcatcgcgggcgtcgaggaaatggtgatcgacatgccggtggaaggctggcgccataccctgttcgccaacctgatcagcaactactcgctgatgcgcaagctggcccccctgatgaaaaagcaaggctccggctatattctgaacgtcagcagctattttggcggcgagaaggacgcggccatcccctacccgaaccgcgccgactatgcggtgagcaaggccggccagcgcgcgatggcggaggtgttcgcgcgcttcctggggccggagattcagatcaacgccattgcccccggcccggtcgagggcgaccggttgcgggggaccggggagcgcccggggctgttcgcccgccgcgcccgcctgatcctggaaaacaaacgcctgaacgaactgcatgccgcgctgattgccgccgcgcggacggacgaacgcagcatgcatgagcttgtcgaactgctgctgccgaacgacgtggccgcgctagagcagaatcccgcagcgcccacggccctgcgcgaactggcccgccggtttcgctccgagggcgatcccgcggcctcctcctccagcgcgctgctgaaccgctcgatagcggcgaaactgctggcccgcctccataacggcggctatgtcctcccggcggacatcttcgcgaacctccctaacccccccgacccgtttttcacccgagcccagatcgaccgggaagcccgcaaggtccgcgacggcatcatgggcatgctgtatctgcagcgcatgcccacagagttcgacgtggcgatggccaccgtgtactacctggccgataggaacgtttccggcgaaacgtttcatccgtcgggcgggctgcggtacgaacgcactcccacgggcggggagctgttcggcctgcccagccccgagcggctggccgagctggtaggcagcacggtgtatctgatcggtgaacacctaaccgagcacctgaacctcctggcgcgggcctatctggagcggtacggcgcccgccaggtagtcatgattgtggaaaccgaaactggcgccgaaaccatgcgccgcctgctgcacgatcacgtggaagcgggccgcctcatgaccatcgtcgcgggcgatcagatcgaggccgcgattgatcaagcgatcacacgctacgggcgccccggccccgtcgtctgcacgcccttccgcccgctgccgaccgtgcccctggtggggcggaaggactccgattggtcaacggtgctgagcgaagccgagttcgccgagctgtgcgagcaccagctgacccaccacttccgtgtggcgcgcaagatcgccctgtcagatggcgccagcctggccctggtgaccccggaaaccaccgccacttccacgacggagcaattcgccctggcgaactttattaaaaccaccctgcacgccttcaccgcgaccattggcgtcgagtcggaacgcaccgcgcagcgcatcctgatcaatcaggtggacctgacccggagagcccgcgccgaggaaccgcgcgatccgcacgagcgacagcaggagctggagcgctttatcgaagccgtcctgctcgtcaccgcgccgcttccgccggaggccgacacgcgctatgcggggcgcattcaccggggccgcgcgataaccgtgtaa

SEQ ID NO: 3 - DNA, 5176 bp, Artificial sequence
  Synthetic
ggccgcgaagacaatggccctgcaggcagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctagaaataattttgtttaactttaggcgcgccatcgtgagaccttgactgcatggtctcatactatcgttgtcttcactagtggatcccccgggctgcaggaattcgatatcaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcacaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccaaaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggc

SEQ ID NO: 4 - protein, 542 aa, Artificial sequence
  Synthetic
MKVITAREAAALVQDGWTVASAGFVGAGHAEAVTEALEQRFLQSGLPRDLTLVYSAGQGDRGARGVNHFGNAGMTASIVGGHWRSATRLATLAMAEQCEGYNLPQGVLTHLYRAIAGGKPGVMTKIGLHTFVDPRTAQDARYHGGAVNERARQAIAEGKACWVDAVDFRGDEYLFYPSFPIHCALIRCTAADARGNLSTHREAFHHELLAMAQAAHNSGGIVIAQVESLVDHHEILQAIHVPGILVDYVVVCDNPANHQMTFAESYNPAYVTPWQGEAAVAEAEAAPVAAGPLDARTIVQRRAVMELARRAPRVVNLGVGMPAAVGMLAHQAGLDGFTLTVEAGPIGGTPADGLSFGASAYPEAVVDQPAQFDFYEGGGIDLAILGLAELDGHGNVNVSKFGEGEGASIAGVGGFINITQSARAVVFMGTLTAGGLEVRAGDGGLQIVREGRVKKIVPEVSHLSFNGPYVASLGIPVLYITERAVFEMRAGADGEARLTLVEIAPGVDLQRDVLDQCSTPIAVAQDLREMDARLFQAGPLHL

SEQ ID NO: 5 - DNA, 1629 bp, Artificial sequence
  Synthetic
atgaaggtgatcaccgcacgcgaagcggcggcactggtgcaggacggctggaccgtggccagcgcgggctttgtcggcgccggccatgccgaggccgtgaccgaggcgctggagcagcgcttcctgcagagcgggctgccgcgcgacctgacgctggtgtactcggccgggcagggcgaccgcggcgcgcgcggcgtgaaccacttcggcaatgccggcatgaccgccagcatcgtcggcggccactggcgctcggccacgcggctggccacgctggccatggccgagcagtgcgagggctacaacctgccgcagggcgtgctgacgcacctataccgcgccatcgccggcggcaagcccggcgtgatgaccaagatcggcctgcacaccttcgtcgacccgcgcaccgcgcaggatgcgcgctaccacggcggcgccgtcaacgagcgcgcgcgccaggccattgccgagggcaaggcatgctgggtcgatgcggtcgacttccgcggcgacgaatacctgttctacccgagcttcccgatccactgcgcgctgatccgctgcaccgcggccgacgcccgcggcaacctcagcacccatcgcgaagccttccaccatgagctgctggcgatggcgcaggcggcccacaactcgggcggcatcgtgatcgcgcaggtggaaagcctggtcgaccaccacgagatcctgcaggccatccacgtgcccggcatcctggtcgactacgtggtggtctgcgacaaccccgccaaccaccagatgacgtttgccgagtcctacaacccggcctacgtgacgccatggcaaggcgaggcagcggtggccgaagcggaagcggcgccggtggctgccggcccgctcgacgcgcgcaccatcgtgcagcgccgtgcggtgatggaactggcgcgccgtgcgccgcgcgtggtcaacctgggcgtgggcatgccggcagcggtcggcatgctggcgcaccaggccgggctggacggcttcacgctgaccgtcgaggccggccccatcggcggcacgcccgcggatggcctcagcttcggtgcctcggcctacccggaggcggtggtggatcagcccgcgcagttcgatttctacgagggcggcggcatcgacctggccatcctcggcctggccgagctggatggccacggcaacgtcaatgtcagcaagttcggcgaaggcgagggcgcatcgattgccggcgtcggcggctttatcaacatcacgcagagcgcgcgcgcggtggtgttcatgggcacgctgacggcgggcgggctggaagtccgcgccggcgacggcggcctgcagatcgtgcgcgaaggccgcgtgaagaagatcgtgcctgaggtgtcgcacctgagcttcaacgggccctatgtggcgtcgctcggcatcccggtgctgtacatcaccgagcgcgcggtgttcgagatgcgcgctggcgcagacggcgaagcccgcctcacgctggtcgagatcgcccccggcgtggacctgcagcgcgacgtgctcgaccagtgctcgacgcccatcgccgttgcgcaggacctgcgcgaaatggatgcgcggctgttccaggccgggcccctgcacctgtaa

SEQ ID NO: 6 - protein, 630 aa, Artificial sequence
  Synthetic
MTASHAVHARSLADPEGFWAEQAARIDWETPFGQVLDNSRAPFTRWFVGGRTNLCHNAVDRHLAARASQPALHWVSTETDQARTFTYAELHDEVSRMAAILQGLDVQKGDRVLIYMPMIPEAAFAMLACARIGAIHSVVFGGFASVSLAARIEDARPRVVVSADAGSRAGKVVPYKPLLDEAIRLSSHQPGKVLLVDRQLAQMPRTEGRDEDYAAWRERVAGVQVPCVWLESSEPSYVLYTSGTTGKPKGVQRDTGGYAVALATSMEYIFCGKPGDTMFTASDIGWVVGHSYIVYGPLLAGMATLMYEGTPIRPDGGILWRLVEQYKVNLMFSAPTAIRVLKKQDPAWLTRYDLSSLRLLFLAGEPLDEPTARWIQDGLGKPVVDNYWQTESGWPILAIQRGIEALPPKLGSPGVPAYGYDLKIVDENTGAECPPGQKGVVAIDGPLPPGCMSTVWGDDDRFVRTYWQAVPNRLCYSTFDWGVRDADGYVFILGRTDDVINVAGHRLGTREIEESLSSNAAVAEVAVVGVQDALKGQVAMAFCIARDPARTATAEARLALEGELMKTVEQQLGAVARPARVFFVNALPKTRSGKLLRRAMQAVAEGRDPGDLTTIEDPGALEQLQAALKG

SEQ ID NO: 7 - DNA, 1893 bp, Artificial sequence
  Synthetic
atgacggcaagccatgccgtgcatgcccgttcgctggccgaccccgaggggttctgggccgaacaggcggcgcgcatcgactgggaaaccccgttcggccaggtgctcgacaacagccgcgcgccctttacgcgctggttcgtcggcgggcgcaccaacctgtgccacaacgcggtcgaccgccacctggcggcccgcgccagccagccggcgctgcactgggtctcgaccgagaccgaccaggcccgcacctttacctacgccgagctgcacgacgaagtcagccgcatggccgcgatcctgcagggcctggacgtgcagaagggcgaccgcgtgctgatctacatgccgatgatcccggaagccgcctttgccatgctggcctgcgcgcgcatcggcgcgatccattcggtggtgttcggcggctttgcctcggtcagcctggccgcgcgcatcgaggatgcccggccgcgcgtggtggtcagcgccgacgccggctcgcgtgccggcaaggtggtgccctacaagccgctgctggacgaggccatccggctctcgtcgcaccagcccgggaaggtgctgctggtggaccggcaactggcgcaaatgccccgtaccgagggccgcgatgaggactacgccgcctggcgcgaacgcgtggccggcgtgcaggtgccgtgcgtgtggctggaatcgagcgagccgtcgtacgtgctatacacctccggcaccaccggcaagcccaagggcgtgcagcgcgataccggcggctacgcggtggcgctggccacctcgatggaatacatcttctgcggcaagcccggcgacaccatgttcaccgcgtcggacatcggctgggtggtggggcacagctatatcgtctacggcccgctgctggccggcatggccacgctgatgtatgaaggcacgccgatccgccccgacggtggcatcctgtggcggctggtggagcaatacaaggtcaacctgatgttcagcgcgccgaccgcgatccgcgtgctgaagaagcaggacccggcctggctgacccgctacgacctgtccagcctgcgcctgctgttcctggccggcgagccgctggacgagcccaccgcgcgctggatccaggacggcctgggcaagcccgtggtcgacaactactggcagaccgaatccggctggccgatcctcgcgatccagcgcggcatcgaggcgctgccgcccaagctgggctcgcccggcgtgcccgcctacggctatgacctgaagatcgtcgacgagaacaccggcgctgaatgcccgccggggcagaagggtgtggtcgccatcgacggcccgctgccgccgggatgcatgagcacggtctggggcgacgacgaccgcttcgtgcgcacctactggcaggcggtgccgaaccggctgtgctattcgaccttcgactggggcgtgcgcgacgccgacggctatgtttttatcctgggccgcaccgacgacgtgatcaacgttgccggccaccggctgggcacccgcgagatcgaggaaagcctgtcgtccaacgctgccgtggccgaggtggcggtggtgggcgtgcaggacgcgctcaaggggcaggtggcgatggccttctgcatcgcccgcgatccggcgcgcacggccacggccgaagcgcggctggcattggagggcgagttgatgaagacggtggagcagcaactgggtgccgtggcgcggccggcgcgcgtattctttgtcaatgcactgcccaagacccgctccggcaagttgctgcggcgcgccatgcaggcggtggccgaagggcgcgatccgggcgacctgaccacgatcgaggacccgggtgcgctggaacagttgcaggcagcgctgaaaggctag

SEQ ID NO: 8 - protein, 576 aa, Artificial sequence
  Synthetic
MAAAALPASRRDDYRALYESFRWEIPPHFNIAEACCGRWARDPATMDRIAVYTEHEDGRRNAHTFAHIQAEANRLSAALRALGVARGDRVAIVMPQRIETVIAHMAIYQLGAIAMPLSMLFGPEALAYRIAHSEANVAIADETSIDNVLAARPECPTLATVIAAGGAHGRGDHDWDVLLAAQLPTFVAEQTKADEAAVLIYTSGTTGPPKGALIPHRALIGNLTGFVCSQNWYPQDDDVFWSPADWAWTGGLWDALMPALYFGKPIVGYQGRFSAERAFELLERYAVTNTFLFPTALKQMMKACPEPRQRYDIRLRALMSAGEAVGETVFGWCRDALGVIVNEMFGQTEINYIVGNCTAQNDDKQLGWPARPGSMGRPYPGHRVQVIDDEGQPCAPGEDGEVAVCATDSAGHPDPVFFLGYWKNEAATAGKYAERDGLRWCRTGDLARVDADGYLWYQGRADDVFKSSGYRIGPSEIENCLLKHPAVSNCAVVPSPDPERGAVVKAFVVLTPSVARSFDGDAALVTELQAHVRGQLAPYEYPKAIEFIDQLPMTTTGKIQRRVLRLLEEARAGKRA

SEQ ID NO: 9 - DNA, 1731 bp, Artificial sequence
  Synthetic
atggccgcagctgcgttgccggcaagccggcgcgacgactatcgcgccctgtatgaatccttccgctgggaaatccccccgcatttcaatatcgccgaggcctgctgcgggcgctgggcgcgcgacccggccacgatggaccgcatcgcggtctataccgagcatgaggacggccgccgcaacgcgcatacctttgcccatatccaggccgaagccaaccgcctgtcggcggcgctgcgcgcactgggcgtggcgcgcggcgaccgcgtggcaatcgtgatgccgcagcggatcgagaccgtgatcgcgcatatggcgatctaccagctcggcgccatcgccatgccgctgtcgatgctgttcgggcccgaggcgctggcctaccgtatcgcacacagcgaagccaatgtggcgatcgcggacgagacttccatcgacaatgtgctggccgcgcgcccggaatgcccgacgctggccaccgtgattgccgccggcggcgcgcatggccgcggcgaccacgactgggacgtgctgctggccgcgcagctgccgacttttgtcgccgagcagaccaaggccgacgaggccgcggtgctgatctacaccagcggcaccaccggcccgcccaagggcgcgctgatcccgcaccgcgcgctgatcggcaacctgaccggctttgtctgctcgcagaactggtatccgcaggacgacgacgtgttctggagcccggccgactgggcctggaccggcggcctgtgggatgcgctgatgccggcgctgtatttcggcaagcccatcgtcggctaccagggccgcttctccgccgagcgcgccttcgagctgctggagcgctacgccgtcaccaacaccttcctgttcccgaccgcgctcaagcagatgatgaaggcctgccccgagccgcggcagcgctacgacatcaggctgcgtgcgctgatgagcgccggcgaggccgtgggcgagaccgtgttcggctggtgccgcgatgcgctgggcgtgatcgtcaacgagatgttcggccagaccgagatcaactacatcgtcggcaactgcaccgcgcagaacgacgacaagcagctgggctggccggcacgaccgggctcgatggggcgtccctatccgggccaccgcgtgcaggtgatcgacgacgaaggccagccctgcgcgccgggcgaggacggcgaggtcgcggtatgcgccaccgacagcgccgggcatccggacccggtgttcttcctcggctactggaagaacgaagccgccaccgcgggcaagtacgccgagcgcgacggcctgcgctggtgccgcaccggcgacctggcgcgcgtcgatgccgatggctacctgtggtaccaggggcgtgccgacgatgtgttcaagtcctcgggctaccgcatcgggccgagcgagatcgagaactgcctgctcaagcatccggcggtgtccaactgcgccgtggtgccctcgcccgaccccgagcgcggcgccgtggtcaaggccttcgtggtgctgacaccgtcggtggcgcgctcgttcgacggcgacgcggcgctggtcacggagctgcaggcgcatgtgcgcggccagctggcgccgtatgaatacccgaaggcgatcgaattcatcgaccagctgccgatgaccaccaccggcaagatccagcggcgcgtgctgcgcttgctggaggaagcgcgcgcgggcaagcgcgcctag

SEQ ID NO: 10 - protein, 685 aa, Artificial sequence
  Synthetic
MSEGKAPRHAAQQELADVSEAEIAVHWPEEDYVPPAGQFIAQANLTDPHIFERFSLERFPECFKEFADLLDWYKYWETTLDTSNPPFWRWFVGGRINACHNCVDRHLAAYRNKTAIHFVPEPEDEAVHHLTYQELFVRVNELAALLREFCGLKAGDRVTLHMPMVAELPITMLACARIGVIHSQVFSGFSGKACAERIADSESRLLITMDAYHRGGELLDHKEKADIAVAEAASAGQQVEKVLIWQRYPGKYSSAALLVKGRDVILNDVLAGFRGRRVEPEPMPAEAPLFLMYTSGTTGRPKGCQHSTGGYLSYVAWTSKYIQDIHPEDVYWCMADIGWITGHSYIVYGPLALAASSVVYEGVPTWPDAGRPWRIAESLGVNIFHTSPTAIRALRRNGPDEPAKYDCHFKHMTTVGEPIEPEVWKWYHREVGKGEAVIVDTWWQTENGGFLCSTLPGIHPMKPGSTGPGIPGIHPVIFDEEGNEVPAGSGKAGNICIRNPWPGIFQTVWKDPDRYVRQYYARYCKNPDSKDWHDWPYMAGDGAMQAADGYFRILGRIDDVINVSGHRLGTKEIESAALLVPDVAEAAVVPVADEVKGKVPDLYVSLKPGLSPSIKIANKVSAAVVSQIGAIARPHRVVIVPDMPKTRSGKIMRRVLAAISNHQEPGDVSTLANPEVVEKIRELAT

SEQ ID NO: 11 - DNA, 2058 bp, Artificial sequence
  Synthetic
atgtctgaaggcaaagcgccacgccatgctgcccagcaggaattggccgatgtgtccgaggccgaaatcgcggtccattggcccgaggaggactatgtcccgccggccggccagttcattgcgcaggccaatctgaccgatccccatattttcgagcgcttctccctcgaacgtttccccgagtgcttcaaggagttcgcagacctgctggactggtacaaatactgggaaacgaccctggataccagcaacccgcctttctggcgctggttcgtcggcggcaggatcaacgcctgccacaattgcgtggatcgccacctcgctgcatacaggaacaagaccgcgattcatttcgtgcccgagccggaggatgaggcggtgcatcacctcacctaccaggagctcttcgttcgcgtcaatgagctggccgccctgctgcgcgagttctgcggcctgaaggccggcgaccgcgtcacgctgcatatgccgatggtggccgaactgcccatcaccatgctcgcctgcgcccgcatcggcgtgattcattcgcaggtattcagcggcttcagcggcaaggcctgcgccgagcgcatcgcggactccgagagccggctgctgatcaccatggacgcctatcaccgcggcggtgaattgctcgatcacaaggaaaaggccgacatcgccgtggcagaagccgccagcgccggtcagcaggtcgagaaggtcctgatctggcagcgctacccgggcaagtattccagtgccgccctactggtgaagggccgcgatgtcattctcaatgacgtgctcgccgggttccgcggcaggcgtgtcgagcccgagccgatgccggcggaggcgccgctgttcctgatgtacacgagcggcaccacgggccggcccaagggctgccagcattccactggcggctatctgtcctatgtggcgtggacctctaagtacatccaggatatccaccccgaggacgtctactggtgcatggccgatattggctggatcaccgggcattcctacatcgtctatggcccgctcgcgctcgccgcttcgtctgtcgtctatgaaggcgtgccgacctggcccgacgccggccggccctggcgtattgcggaaagccttggcgtcaatatcttccacacctcgcccaccgcaatccgcgcgctgcggcgcaacgggcccgacgagccggcgaagtacgactgccatttcaagcacatgaccacggtgggcgagccgatcgagcccgaagtctggaagtggtaccaccgtgaagtcggcaaaggcgaggcggtgatcgtggacacctggtggcaaaccgagaatggcggcttcctctgcagcacgctgccgggcatccacccgatgaagcccggcagcactggcccgggaatcccgggcattcatccggtgatctttgacgaggaaggcaatgaggtcccggccggctcgggcaaggcgggcaacatctgcatccgcaatccctggccgggcatattccagaccgtctggaaggatccggaccgctacgtgcgccagtactatgcgcgctattgcaagaatcccgacagcaaggactggcacgactggccgtatatggcgggcgatggcgcaatgcaggcggcggacggctactttcgcatccttggccgcatcgacgacgtgatcaatgtttccggccatcgcctcggcaccaaggagatcgaatccgcagcactgctggtgccggacgtcgccgaggcggcggtggtgccggtggccgacgaggtcaagggcaaggtgcctgatctctatgtatcgctcaagccgggactgtcgccctccatcaagatcgcgaacaaggtctcggccgcggtggtatcccagattggcgcgattgcgcgtccgcatcgggtcgtgatcgtccccgacatgcccaagacacgctcgggcaagatcatgcgccgcgtgctggcggcgatctccaaccaccaggagcctggcgacgtatccacgcttgccaatccggaggtcgtcgagaagatcagggagctggcgacatag

SEQ ID NO: 12 - protein, 660 aa, Artificial sequence [sequence omitted]

SEQ ID NO: 13 - DNA, 1983 bp, Artificial sequence
  Synthetic
atgtccgccatcgaatcggtgatgcaagagcatcgcgtgttcaacccgcccgaaggcttcgccagccaggccgcgatccccagcatggaggcctaccaggcgctgtgcgacgaagccgagcgtgactatgaaggtttctgggcgcgccacgcgcgcgagctgctgcactggaccaagcccttcaccaaggtgctggaccaaagcaacgcaccgttctacaagtggttcgaagacggcgagctcaacgcctcttacaactgcctggaccgcaatctgcagaacggcaatgcggacaaggtcgcgatcgtgttcgaggccgacgacggcagcgtgacgcgcgtcacctaccgcgagctgcatggcaaggtgtgccgcttcgccaacggcctgaaggcgctcggcatcaggaagggcgaccgcgtggtgatctacatgccgatgtcggtcgaaggcgtggtcgcgatgcaggcctgcgcacgcctgggcgccacgcactcggtggtgttcggcggcttctcggccaagtcgctgcaggagcggctggtggacgtgggcgcggtggcgctgatcaccgccgacgagcagatgcgcggcggcaaggcgctgccgctcaaggccatcgccgatgacgcgctggcgctgggcggctgcgaggccgtcaggaacgtgatcgtctaccgccgcaccggcggcaaggttgcctggaccgaaggccgcgaccgctggatggaagatgtcagcgccggccagccggatacctgcgaagccgagccggtgagcgccgagcacccgctgttcgtgctctacacctccggctccaccggcaagcccaagggcgtgcagcacagcaccggcggctacctgctgtgggcgctgatgacaatgaagtggaccttcgacatcaagcccgacgacctgttctggtgtaccgcggacatcggctgggtcaccggccacacctatattgcctacggcccgctggccgcgggcgccacccaggtggtgttcgaaggcgtgccgacctaccccaacgccggccgcttctgggacatgatcgcgcgccacaaggtcagcatcttctacaccgcgccgaccgcgatccgctcgctgatcaaggccgccgaggccgacgagaagatccacccgaaacagtacgacctgtccagcctgcgcctgctcggcaccgtgggcgagccgatcaaccccgaagcctggatgtggtactacaagaacatcggcaacgagcgctgcccgatcgtcgacaccttctggcagaccgagaccggcggccacatgatcacgccgctgccgggcgcgacgccgctggtgccgggttcgtgcacgctgccgctgccgggcatcatggccgccatcgtcgacgagaccggccatgacgtgcccaacggcaacggcggcatcctggtggtcaagcgtccgtggccggccatgatccgcaccatctggggcgatccggagcgcttcaggaagagctacttccccgaagagctcggcggcaagctctacctggccggcgacggctcgatccgcgacaaggacaccggctacttcaccatcatgggccgcatcgacgacgtgctgaacgtgtcgggccaccgcatggggacgatggagatcgagtccgcgctggtgtccaacccgctggtggctgaagccgccgtggtgggccgccccgacgacatgaccggcgaggccatctgcgccttcgtcgtgctcaagcgttcgcgtccgactggcgaagaggccgtcaagatcgcgacggagctgcgcaactgggtcggcaaggagatcggcccgatcgccaagcccaaggacatccgctttggcgacaacctgcccaagacgcgctcgggcaagatcatgcggcgcctgctgcggtcgctggccaagggggaggagatcacgcaggacacctcgacgctggagaatccggccatcctggagcagctcaagcaggcgcagtga

SEQ ID NO: 14 - protein, 714 aa, Artificial sequence
  Synthetic
MSTRDLYTHAQLRRLFHPRTIAVVGATPNARSFAGRAMTNLQQFDGNVLLVNPRYPEVNGQVCYPSLSALPEAPDCVLIATARETVEPIVRECAGLGVGGVVLFASGYAETGNPEQIAEQARLVAIARESGMLLLGPNSIGYANYINHALVSFTPLPARGGELPAHAIGLVSQSGALAFALEQAANHGTAFSHVFSCGNACDIDVTDQIAYLAGDPSCAAIACVFEGLSDASRIIRAAQVCAEAGKPLVVYKMARGTAGAAAAMSHTGSMAGSDRAYSTALREAGVVQVDTIEQLVPTTVFFAKAPRPTTSGVAIVSGSGGAGIVAADEAERFNVPLPQPCDATRAVLESHIPDFGAARNPCDLTAQAANNFDSFIQCGDAVFADPAYGAAVVPLVVTGDGNGRRFQVFNDLAVKHGKMACGLWMSNWMEGPEAVESEALPRLALFRSVSHCFAALAAWQAREQWLLSRATPKPPRLTHASVAAEARARIVAAPADTLTEREAKDVLAMYGVPVVGESLATSEQDAVRAADACGYPVVLKVESPAIPHKSEAGVIRLGVNSAQEVAVAYREVMANARKVTADDRINGVLVQSQVPTGIEILVGARVDPHLGALLVVGLGGVMVELMQDTVATIAPCSAQQARAMLEQLRGVALLKGFRGAAGVDMDLLAEIVASLSEFAADQRDVIAEFDVNPLICTPDRIVAVDALIERRVGA

SEQ ID NO: 15 - DNA, 2145 bp, Artificial sequence
  Synthetic
atgtcgacacgcgatctctatacccacgcgcaactgcggcgcctcttccatccgcgcaccatcgcggtggtcggcgcgacgccgaacgctcgctcgttcgccggccgggccatgacgaacctgcagcagttcgacggcaacgtgctgctggtcaacccccgctaccccgaggtgaacgggcaggtctgctatccgtcgctgtcggcgctgcccgaggcgcccgactgcgtgctgatcgccaccgcgcgcgaaacggtggagcccatcgtgcgcgagtgcgcggggctgggcgtgggcggcgtggtgctgttcgcgtcgggctatgccgagaccggcaatccggagcagattgccgagcaggctcggctggtcgccattgcccgggaaagcggcatgctgctgctcggtccgaacagcatcggctatgcgaactacatcaaccatgcgctggtgtcgttcacgccgctgcccgcgcgtggcggcgaactgccggcccatgcgatcgggctggtcagccagtccggcgcgctggcatttgcgctggaacaggcggccaaccacggcacggcgttcagccacgtgttctcgtgcggcaatgcgtgcgatatcgacgtgaccgaccagatcgcctatctcgccggggatccctcgtgcgcggcgatcgcatgcgtattcgaagggctgtccgacgccagccggatcattcgcgcggcgcaagtctgcgcggaagccggcaagccgctggtggtctacaagatggcgcgcgggacggcgggcgcggcggcggccatgtcgcataccggctcgatggcgggatccgaccgcgcctacagcacggcgctgcgcgaagctggcgtggtgcaggtcgataccatcgagcagctcgtgccgacgacggtgttcttcgccaaggccccccggccgacgacgtccggcgtggccatcgtctcgggttcgggcggcgcgggcattgtcgccgccgacgaggccgagcgtttcaacgtgccgctgccgcagccgtgtgacgcgacccgcgccgtgctcgaatcgcacattcctgacttcggcgccgcgcgcaacccgtgcgacctgaccgcccaggccgccaacaacttcgactccttcatccagtgcggcgacgcggtcttcgccgatcccgcctacggcgccgccgtggtgccgctggtggtgaccggcgacggcaacggccgccgcttccaggtgttcaacgacctagccgtcaagcacggcaagatggcgtgcggcctgtggatgtcgaactggatggaagggccggaggcggtcgagtccgaggcgctgccgcgccttgcgctgttccgctcggtctcgcactgcttcgcggcgctggccgcgtggcaggcacgggagcaatggctgttgtcgcgcgccacgccgaagccgccgcgcctgacacacgcttcggtggccgccgaagcgcgcgcgcgcatcgttgccgcgccggccgatacgctcaccgagcgtgaagccaaggacgtccttgccatgtacggcgtgccggtggtgggcgagtccctggcgacgagcgagcaggacgccgtgcgcgccgccgatgcctgcggctatccggtcgtgctgaaggtcgagagcccggccatcccgcacaagtcggaagcgggcgtgatccgcctcggcgtgaactcggcgcaggaggttgccgtcgcgtaccgcgaggtcatggcgaatgcgcgcaaggtgaccgccgacgaccgcatcaacggcgtgctggtgcagagccaggtgccgaccggcatcgagatcttggtcggcgcccgcgtggacccgcacctcggcgcgctgctggtggtggggctgggcggggtgatggtcgagctgatgcaggacacggtcgcgaccatcgcgccgtgctcggcgcagcaggcgcgcgccatgctggagcagctgcgcggcgtggcgctgctgaagggcttccgcggcgcggcgggcgtggacatggacctgctggcggaaatcgtcgccagcctgtccgagttcgcggcggaccagcgcgacgtgatcgccgagttcgatgtgaatccgctgatctgcacgccggaccgcatcgtggcggtggatgcgctgatcgaacggagagtgggggcctga

SEQ ID NO: 16 - protein, 660 aa, Artificial sequence
  Synthetic
MTSIQSVVHEGRMFPPSRHASAKAAIPSMEAYQALCDEAERDYEGFWARHARELLHWTKPFTKVLDQSNAPFYKWFEDGELNASYNCLDRNLQNGNADKVAIVFEADDGSVTRVTYRELHGKVCRFANGLKALGIRKGDRVVIYMPMSVEGVVAMQACARLGATHSVVFGGFSAKSLQERLVDVGAVALITADEQMRGGKALPLKPIADDALALGGCEAVRNVIVYRRTGGKVAWTEGRDRWMEDVSAGQPETCEAEPVSAEHPLFVLYTSGSTGKPKGVQHSTGGYLLWALMTMKWTFDIKPDDLFWCTADIGWVTGHTYIAYGPLAAGATQVVFEGVPTYPNAGRFWDMIARHKVSIFYTAPTAIRSLIKAAEADEKIHPKQYDLSSLRLLGTVGEPINPEAWMWYYKNIGNERCPIVDTFWQTETGGHMITPLPGATPLVPGSCTLPLPGIMAAIVDETGHDVPNGNGGILVVKRPWPAMIRTIWGDPERFRKSYFPEELGGKLYLAGDGSIRDKDTGYFTIMGRIDDVLNVSGHRMGTMEIESALVSNPLVAEAAVVGRPDDMTGEAICAFVVLKRSRPTGEEAVKIATELRNWVGKEIGPIAKPKDIRFGDNLPKTRSGKIMRRLLRSLAKGEEITQDTSTLENPAILEQLGQAR

SEQ ID NO: 17 - DNA, 1983 bp, Artificial sequence
  Synthetic
atgacaagcattcaatccgttgtgcacgaagggcggatgttcccgccatcccgccacgccagcgctaaggccgcgattcccagcatggaggcctaccaggcactgtgcgacgaagccgagcgtgactatgaaggtttctgggcgcgccacgcgcgcgagctgctgcactggaccaagcccttcaccaaggtgctggaccaaagcaacgcaccgttctacaagtggttcgaagacggcgagctcaacgcctcttacaactgcctggaccgcaatctgcagaacggcaatgcggacaaggtcgcgatcgtgttcgaggccgacgacggcagcgtgacgcgcgtcacctaccgcgagctgcatggcaaggtgtgccgctttgccaacggcctgaaggcgctcggcatcaggaagggcgaccgcgtggtgatctacatgccgatgtcggtcgaaggcgtggtcgcgatgcaggcctgcgcacgcctgggcgccacgcactcggtggtgttcggcggcttctcggccaagtcgctgcaggagcggctggtggacgtgggcgcggtggcgctgatcaccgccgacgagcagatgcgcggcggcaaggcgctgccgctcaagcccatcgccgatgacgcgctggcgctggggggctgcgaggccgtcaggaacgtgatcgtctaccgccgcaccggcggcaaggttgcctggaccgaaggccgcgaccgctggatggaagatgtcagcgccggccagccggagacctgcgaagccgagccggtgagcgccgagcacccgctgttcgtgctctacacctccggctccaccggcaagcccaagggcgtgcagcacagcaccggcggctacctgctgtgggcgctgatgacaatgaagtggaccttcgacatcaagcccgacgacctgttctggtgtaccgcggacatcggctgggtcaccggccacacctatattgcctacggcccgctggccgcgggcgccacccaggtggtgttcgaaggcgtgccgacctaccccaacgccggccgcttctgggacatgatcgcgcgccacaaggtcagcatcttctacaccgcgccgaccgcgatccgctcgctgatcaaggccgccgaggccgacgagaagatccacccgaaacagtacgacctgtccagcctgcgcctgctcggcaccgtgggcgagccgatcaaccccgaagcctggatgtggtactacaagaacatcggcaacgagcgctgcccgatcgtcgacaccttctggcagaccgagaccggcggccacatgatcacgccgctgccgggcgcgacgccgctggtgccgggttcgtgcacgctgccgctgccgggcatcatggccgccatcgtcgacgagaccggccatgacgtgcccaacggcaacggcggcatcctggtggtcaagcgtccgtggccggccatgatccgcaccatctggggcgatccggagcgcttcaggaagagctacttccccgaagagctcggcggcaagctctacctggccggcgacggctcgatccgcgacaaggacaccggctacttcaccatcatgggccgcatcgacgacgtgctgaacgtgtcgggccaccgcatggggacgatggagatcgagtccgcgctggtgtccaacccgctggtggccgaagccgccgtggtgggccgccccgacgacatgaccggcgaggccatctgcgccttcgtcgtgctcaagcgttcgcgtccgactggcgaagaggccgtcaagatcgcgacggagctgcgcaactgggtcggcaaggagatcggcccgatcgccaagcccaaggacatccgctttggcgacaacctgcccaagacgcgctcgggcaagatcatgcggcgcctgctgcggtcgctggccaagggggaggagatcacgcaggacacctcgacgctggagaatccggccatcctggagcagcttggccaggcacgctga

SEQ ID NO: 18 - protein, 550 aa, Artificial sequence
  Synthetic
MRDYAQAFDGFSYDDAVARQLHGSQEAMNACVECCDRHALPGRIALFWEGRDGNSRSWTFTELQALSAQFAGFLKAQGVQPGDRVAGLLPRNAELLVTILGTWRAGAVYQPLFTAFGPKAIEHRLNASGAKVVVTDGANRPKLDDVDGCPAIVTVAGDKGRGLVRGDFSFWAELERQPASFEPVPRRGDDPFLMMFTSGTTGPAKPLLVPLKAIAAFAGYMSDAVDLRAEDAFWNLADPGWAYGLYYAVTGPLALGHPTTFYDGPFTVESTCRVIRKYGITNLAGSPTAYRLLIAAGEAVSGPLRGRLRAVSSAGEPLNPEVIRWFASELGVTIHDHYGQTELGMVLCNHHALAHPVRMGAAGFASPGHRVVVVDDEQRELPPGRPGTLALDLKRSPMCWFGGYHGTPTSGFAGGYYLTGDSAELNDDGSISFIGRADDVITTSGYRVGPFDVESALIEHPAVVEAAVIGKPDPERTELIKAFVVLDPQYRAAPELAEALRQHVRKRLAAHAYPREIEFVVELPKTPSGKVQRFILRNQEVARAREAAAA

SEQ ID NO: 19 - DNA, 1653 bp, Artificial sequence
  Synthetic
atgcgcgactacgcccaagccttcgacggattttcctatgacgacgccgtggcacggcaactgcacggcagccaggaggcaatgaacgcctgcgtcgaatgctgcgaccgccacgcgctgccgggccgtatcgcgctgttctgggaagggcgagacggcaattcgcgcagctggacctttaccgagctgcaggcactgtccgcgcagtttgccggcttcctgaaggcgcagggcgtgcagccgggcgaccgcgtggcgggcctgctgccgcgcaatgcggaactgctggtgacgattctcggcacctggcgcgccggcgcggtgtaccagccgctgttcacggccttcggccccaaggccatcgagcaccggctcaatgcgtccggcgcgaaggttgtggtcaccgatggcgccaaccgccccaagctggatgacgtggatggctgtcccgccattgtcaccgtggccggcgacaagggccgcggcctggtgcgcggcgacttcagcttctgggccgaactggaacgccagccggcgtcgttcgagccggtgccgcgccggggcgacgaccccttcctgatgatgttcacctccggcaccaccggcccggccaagccgctgctggtgccgctcaaggccattgccgcgtttgccggctatatgagcgacgcggtcgacctgcgcgcggaagacgctttctggaacctggccgatccgggctgggcctatggcctgtattacgcggtcacgggcccgctggcgctgggccatcccaccaccttctacgatggcccgttcaccgtggagagcacatgccgtgtgatccgcaagtacggcatcaccaacctggccggctcgcccacggcataccggctgctgatcgccgcgggcgaggccgtgtcaggcccgctgcgcgggcggctgcgcgcggtcagcagcgcgggcgagccgctcaacccggaagtgatccgctggttcgccagcgagctgggcgtgaccatccacgaccactacggccagaccgagctgggcatggtgctgtgcaaccaccatgcgctggcgcatccggtgcgcatgggcgcggccggctttgccagccccgggcaccgcgtggtggtggtggacgatgaacagcgcgaactgccgccgggccggccgggcacgctggcgctggacctgaagcgctcgccgatgtgctggttcggcggctatcacggcacgcccaccagcgggtttgccggcggctactacctgaccggcgattccgccgagctgaatgacgacggcagcatcagcttcataggccgggccgacgacgtcatcaccacctctggctaccgcgtgggcccgttcgacgtggaaagcgcgctgatcgagcacccggccgtggtcgaggccgcggtgatcggcaagcccgatccggagcgcaccgagctgatcaaggcctttgtcgtgctggacccgcaatatcgcgccgcgccggaactggccgaggcgctgcgccagcacgtgcgtaagcgcctggccgcccatgcctacccgcgcgagatcgagttcgtcgtcgagctgcccaagacccccagcggcaaggtccagcgctttatcctgcgcaaccaggaagtggcccgcgcgcgcgaggcggccgctgcctga